(19) United States
(12) Patent Application Publication
(10) Pub. No.: US 2007/0233027 A1
(43) Pub. Date: Oct. 4, 2007

(54) Absorbent article with sensation member

(75) Inventors: Donald Carroll Roe, West Chester, OH (US); Jennifer Joan Nandrea, Cincinnati, OH (US); Masaharu Nishikawa, Montgomery, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/724,851

(22) Filed: Mar. 16, 2007

Related U.S. Application Data
(60) Provisional application No. 60/788,343, filed on Mar. 31, 2006.

(51) Int. Cl. A61F 13/15

(52) U.S. Cl. 604/361

(57) ABSTRACT

An absorbent article includes an outer cover having a longitudinal axis, a topsheet attached to the outer cover and having a body-facing surface, and an absorbent core disposed between the outer cover and the topsheet. The article may also include barrier leg cuffs. The article further includes a sensation member.

### CROSS REFERENCE TO RELATED APPLICATION

This application claims the benefit of U.S. Provisional Application No. 60/788,343, filed Mar. 31, 2006, the substance of which is incorporated herein by reference.

### FIELD OF THE INVENTION

The present disclosure generally relates to absorbent articles, including diapers, training pants, pull-on diapers, absorbent inserts, diaper holders and liners, and the like, and in particular to an absorbent article with a sensation member, which may be adapted for use in urinary toilet training.

### BACKGROUND OF THE INVENTION

Absorbent articles are well known in the art. These articles typically have an absorbent assembly held or positioned in proximity to the body of a wearer during use in order to capture and absorb bodily exudates discharged from the wearer. Typical absorbent articles include a topsheet facing the wearer, which permits fluid exudates to pass through, and an outer cover, which prevents the exudates from escaping from the absorbent article.

Disposable absorbent articles such as diapers are designed to absorb and contain bodily waste in order to prevent soiling of the body and clothing of the wearer. Disposable diapers typically comprise a single design available in different sizes to fit a variety of wearers ranging from newborns to toddlers undergoing toilet training. The design of the diaper typically affects performance, such as the ability to absorb and contain bodily waste. The fit of the diaper on the wearer's body is typically affected by, for example, the size of the diaper waist opening, the size of the openings around the thighs, and the length or “pitch” of the diaper.

The toilet training stage may be referred to as the “point of exit” from the diaper product category because toddlers who have successfully completed toilet training typically no longer wear diapers. The age at which children are toilet trained in “developed” countries has increased steadily over the past several decades and is now in the range of about 24-48 months. One reason for which toilet training has become delayed is that significant technical improvements have been made in diaper dryness and comfort. For example, when wearing a typical modern diaper, the child may have dry skin even after one or more occurrences of urination. As a result, the child may feel little or no discomfort and often may not even be aware that he or she has urinated.

Some parents may have the child wear cotton training pants or cotton underwear during urinary training so the child feels discomfort following urination in his or her “pants.” It is believed that such discomfort assists with learning or provides motivation to learn to voluntarily retain urine. Cloth training pants leave the skin wet and, due to their high breathability, promote evaporative cooling of the skin, further enhancing discomfort. One tradeoff in this approach, however, is that cloth training pants have poor urine containment, often leading to wet clothing and wet surroundings, e.g., carpeting, furniture, etc.

More recently, disposable absorbent articles that are suitable for urinary training have become available to parents. These absorbent articles include a sensation member provided such that at least a portion of the sensation member is held in close contact with the wearer's skin during use. The sensation member can be a wetness sensation member, a temperature sensation member and/or a tactile sensation member, or any other sensation member that is capable of signaling to the wearer that a discharge of bodily exudates, such as urine, has occurred. In use, it is the portion of the sensation member, which has been wetted and which is in contact with the wearer's skin that provides this signal. The wearer remains aware of the signal/sensation as long as this “signaling” portion remains in contact with the wearer's skin.

Although these disposable articles are an improvement over cloth training pants, the inventors have found that the effectiveness of the signal delivered to the wearer is reduced when the sensation member does not contact the wearer's skin reliably during all child activities or bodily positions.

In addition, the inventors have found that these disposable articles tend to reach an elevated relative humidity of the air inside the article soon after the first insult of urine. The inventors have found in order to deliver an effective signal to the wearer it may beneficial to provide a highly breathable article with a sensation member in order to intensify and/or lengthen the signal delivered to the wearer's skin.

Consequently, there is a need to provide a disposable absorbent article with a sensation member that is capable of delivering an effective signal to a wearer over a prolonged period of time, regardless of the activity or position of the wearer.

### SUMMARY OF THE INVENTION

In one aspect, the invention is directed to a refastenable absorbent article having a waist region and a crotch region, comprising a outer cover wherein the outer cover has a moisture vapor transmission rate of at least 1000 g/m²/24 hr, a sensation member having a body-facing surface, wherein at least a portion of the body facing surface of the sensation member is capable of contacting the skin of a wearer and wherein said portion has a Stiffness of less than about 100 N/m and an absorbent core disposed between the outer cover and the sensation member.

In another aspect, the invention is directed to a refastenable absorbent article having a waist region and a crotch region, comprising a outer cover wherein the outer cover has a moisture vapor transmission rate of at least 1000 g/m²/24 hr, a sensation member having a body-facing surface, wherein at least a portion of the body facing surface of said sensation member is capable of contacting the skin of a wearer and wherein said portion has a Skin Contact Time of at least 30 seconds, and an absorbent core disposed between the outer cover and the sensation member.

In yet another aspect, the invention is directed to a refastenable absorbent article having a waist region and a crotch region, comprising a outer cover wherein the outer cover has a moisture vapor transmission rate of at least 1000 g/m²/24 hr, a sensation member having a body-facing surface, wherein at least a portion of the body facing surface of said sensation member is capable of contacting the skin of a wearer and wherein said portion has a Wet Peel Force of at least 30 grams force, and an absorbent core disposed between the outer cover and the sensation member.

In a further aspect, the invention is directed to an array of absorbent articles each having a sensation member having a body-facing surface, wherein at least a portion of the body facing surface of said sensation members is capable of contacting the skin of a wearer and wherein said portion has a property chosen for at least one of surface area, Contraction Ratio at a 10 g load, force required to elongate a foreshortened region of the sensation member from a relaxed state to 85% of its length in a fully elongated state, Stiffness, Skin Contact Time and Wet Peel Force such that the properties of the each of the absorbent articles forming the array have different values.

In another aspect, the invention is directed to an absorbent article having a waist region and a crotch region, comprising a outer cover wherein the outer cover has a moisture vapor transmission rate of at least 1000 g/m²/24 hr, first and second barrier leg cuffs, at least one of said first and second barrier leg cuffs having an upper edge, a sensation member having a suspended central region having at least a side edge and an absorbent core disposed between the outer cover and the sensation member, wherein the projected height of the side edge is between about 90% and about 300% the projected height of the upper edge of at least one of said first and second barrier leg cuff.

In yet another aspect, the invention is directed to an absorbent article having a waist region and a crotch: region, the article comprising a outer cover, wherein the outer cover has a moisture vapor transmission rate of at least 3500 g/m²/24 hr, a sensation member having a body-facing surface, wherein at least a portion of the body facing surface of the sensation member is capable of contacting the skin of a wearer and wherein the portion has a Stiffness of less than about 100 N/m and wherein the portion of the body facing surface of the sensation member has a Skin Contact Time of at least 30 seconds; and An absorbent core disposed between the outer cover and the sensation member.

Additional aspects of the disclosure are defined by the claims of this patent.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. In the accompanying drawing figures, like reference numerals identify like elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a plan view of an absorbent article with a section of a topsheet removed to expose an underlying absorbent core;

FIG. 2 is a perspective view of an exemplary absorbent article shown in its relaxed, contracted state, i.e., with the contraction induced by elastic members;

FIG. 3a is a plan view of an absorbent article having a sensation member according to an embodiment of the present disclosure;

FIG. 3b is a cross-sectional view of the article shown in FIG. 3a illustrating the layers of the sensation member;

FIG. 4a is an isometric view of the article shown in FIG. 3a illustrating a first exemplary attachment of the sensation member;

FIG. 4b is an isometric view of the article shown in FIG. 3a illustrating a second exemplary attachment of the sensation member;

FIG. 5a is a plan view of an absorbent article having a sensation member according to another embodiment of the present disclosure;

FIG. 5b is a cross-sectional view of the article shown in FIG. 5a illustrating the layers of the sensation member;

FIG. 6a is a plan view of an absorbent article having a sensation member according to a further embodiment of the present disclosure;

FIG. 6b is a cross-sectional view of the article shown in FIG. 6a illustrating the layers of the sensation member;

FIG. 7a is a cross-sectional view of another embodiment of an absorbent article;

FIG. 7b is a cross-sectional view of another embodiment of an absorbent article;

FIG. 7c is a cross-sectional view of another embodiment of an absorbent article;

FIG. 8a is a schematic representation of a Force stand shown with a test sample in a first configuration;

FIG. 8b is a schematic representation of a Force stand shown with a test sample in a second configuration;

FIG. 8c is a schematic representation of a Force stand shown with a test sample in a third configuration;

FIG. 9a is a schematic front view of the components of a plunger;

FIG. 9b is a schematic side view of the components of the plunger shown in FIG. 9a;

FIG. 9c is a schematic front view of a base support platform;

FIG. 9d is a representation of an exemplary force/extension curve; and

FIG. 10 is a cross-sectional view of a preferred embodiment of the absorbent core.

### DETAILED DESCRIPTION OF THE INVENTION

### Definitions

As used herein, the following terms have the following meanings:

The term “absorbent article” refers to a device that absorbs and contains liquid, and more specifically, refers to a device that is placed against or in proximity to the body of the wearer to absorb and contain the various exudates discharged from the body.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods may be used to attach elements together over a particular area either continuously or intermittently.

The term “caregiver” refers to a person other than the child, such as, a parent, babysitter, family member, teacher, day care worker, or other person who is able to provide sufficient assistance to the child to complete a personal hygiene task.

The term “diaper” refers to an absorbent article generally worn by infants and incontinent persons about the lower torso and having the general form of a sheet, different portions of which are fastened together to encircle the waist and the legs of the wearer.

The term “disposable” refers to absorbent articles that generally are not intended to be laundered or otherwise restored or reused as absorbent articles, i.e., they are intended to be discarded after a single use and, preferably, to be recycled, composted or otherwise disposed of in an environmentally compatible manner.

The term “disposed” is used to mean that an element(s) is formed (joined and positioned) in a particular place or position as a unitary structure with other elements or as a separate element joined: to another element.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower” and “top” and “bottom”, respectively.

The term “lateral” or “transverse” refers to a direction running at a 90 degree angle to the longitudinal direction and includes directions within ±45° of the lateral direction.

The term “longitudinal” refers to a direction running parallel to the maximum linear dimension of the article and includes directions within ±45° of the longitudinal direction.

The term “refastenable” refers to the property of two elements being capable of releasable attachment, separation, and subsequent releasable reattachment without substantial permanent deformation or rupture.

The terms “releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

The term “toilet training” refers to the development of continence, which is the ability to voluntarily retain one's urine and feces. Individuals who are incontinent are unable to voluntarily retain their bodily discharges and, instead, urinate and defecate reflexively. For example, newborn babies are incontinent. Coincident with the development of continence, children typically develop the ability to voluntarily urinate and defecate, and cease reflexive elimination. This development of continence and of voluntary elimination, in place of reflexive elimination, may be accelerated and/or guided by caregivers through associative and conditioning techniques of training the child. For the purpose of the present disclosure, the term “toilet training” is used to denote training both for continence, itself, and for the voluntary elimination that is associated with continence. It is also noted that the term “toilet training” is synonymous with the term “potty training”.

The term “training pants” refers to an absorbent article generally worn by infants and incontinent persons about the lower torso and having the general form of a pair of short pants that can be applied or removed from the wearer without unfastening.

The term “unitary” refers to an absorbent article that is formed of separate parts united together to form a coordinated entity so as to not require separate manipulative parts like a separate holder and liner.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

The term “x-y plane” refers to the generally planar structure of a sheet material defined by its length and width and lies between the sheet material's two major surfaces regardless of whether or not the sheet material is flat or curved.

The term “z-direction” refers to the direction through the thickness of a sheet material and generally orthogonal to the x-y plane.

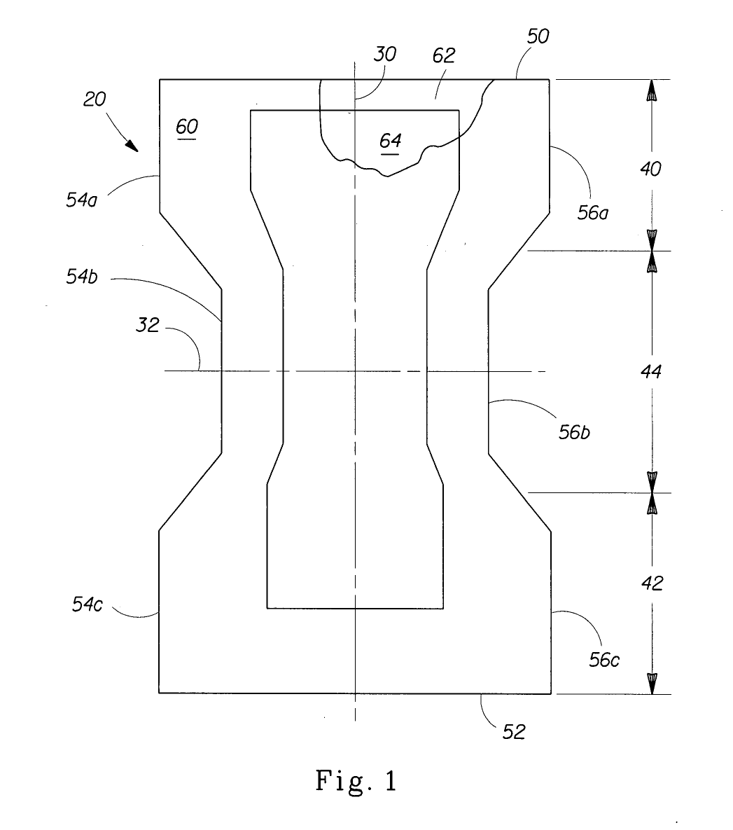

FIG. 1 is a plan view of an exemplary disposable absorbent article 20 in its flat, uncontracted state, i.e., without elastic-induced contraction. Portions of the article 20 have been cut away to more clearly show the underlying structure of the disposable absorbent article 20. As illustrated, the portion of the disposable absorbent article 20 that contacts the wearer faces the viewer (i.e., showing the interior or inner side of the article). The disposable absorbent article 20 has a longitudinal axis 30 and a transverse axis 32.

One end portion of the disposable absorbent article 20 is configured as a first waist region 40 of the disposable absorbent article 20. The opposite end portion is configured as a second waist region 42 of the disposable absorbent article 20. The waist regions 40 and 42 generally comprise those portions of the disposable absorbent article 20 which, when worn, encircle the waist of the wearer. The waist regions 40 and 42 may include elastic elements such that they gather about the waist of the wearer to provide improved fit and containment. An intermediate portion of the disposable absorbent article 20 is configured as a crotch region 44, which extends longitudinally between the first and second waist regions 40 and 42. The crotch region 44 is that portion of the disposable absorbent article 20 which, when the disposable absorbent article 20 is worn, is generally positioned between the legs of the wearer.

The disposable absorbent article 20 has a laterally extending first waist edge 50 in the first waist region 40 and a longitudinally opposing and laterally extending second waist edge 52 in the second waist region 42. The disposable absorbent article 20 has a first side edge 54 and a laterally opposing second side edge 56, both side edges extending longitudinally between the first waist edge 50 and the second waist edge 52. The portion of the first side edge 54 in the first waist region 40 is designated 54a, the portion in the crotch region 44 is designated 54b, and the portion in the second waist region 42 is designated 54c. The corresponding portions of the second side edge 56 are designated 56a, 56b, and 56c, respectively.

The disposable absorbent article 20 preferably comprises a water-permeable topsheet 60, a water-impermeable outer cover 62, and an absorbent assembly or core 64, which may be disposed between the topsheet 60 and the outer cover 62 with the topsheet 60 attached to the outer cover 62. In some embodiments, the absorbent assembly or core may comprise a fluid acquisition component, a fluid distribution component, and a fluid storage component. An example of a suitable absorbent core having a fluid acquisition component, a fluid distribution component, and a fluid storage component is described in U.S. Pat. No. 6,590,136. The topsheet 60 may be fully or partially elasticized or may be foreshortened so as to provide a void space between the topsheet 60 and the core 64. As explained below, a fully or partially elasticized topsheet 60 may also to tend to draw a sensation member against the skin of the wearer. Exemplary structures including elasticized or foreshortened topsheets are described in greater detail in U.S. Pat. Nos. 4,892,536, 4,990,147, 5,037,416, and 5,269,775, among others.

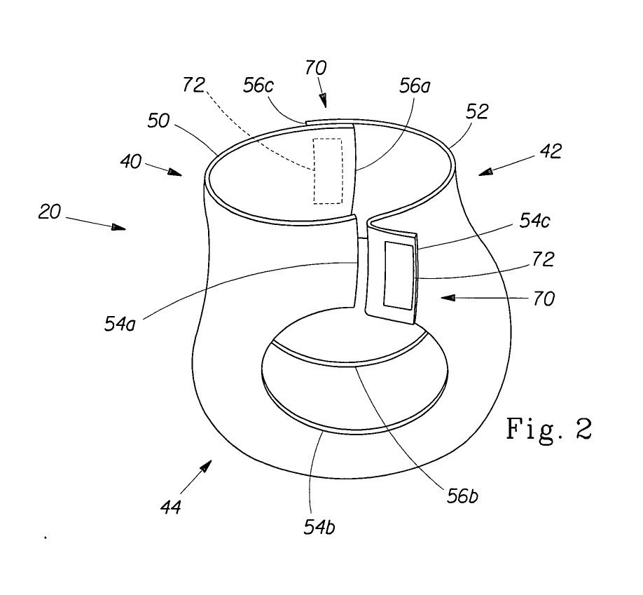

FIG. 2 illustrates the article illustrated in FIG. 1 configured to as it would be worn. The disposable absorbent article 20 may be sealed at the sides so as to be configured as illustrated in FIG. 2. However, the article 20 may instead include refastenable side seams 70 that can be used to fasten the waist regions 40, 42 together. According to one exemplary embodiment, the waist regions 40, 42 may be fastened at the sides to apply the article like a diaper. According to a further exemplary embodiment, illustrated in FIG. 2, the side seams 70 may include fasteners 72 that can be used to configure the article like a pair of pull-on training pants or disposable pants.

As illustrated, the fasteners 72 may be disposed on the interior of the disposable absorbent article 20 in the second waist region 42 adjacent to the portion 54c of the first side edge 54 and adjacent to the portion 56c of the second side edge 56. The portion 54c of the side edge 54 is shown in an open condition, such as prior to closing and fastening or after being reopened. The portion 56c of the opposing side edge 56 is shown fastened, i.e., forming a pants configuration. In FIG. 2, the second waist region 42 overlaps the first waist region 40 when they are fastened together.

The fasteners 72 may be formed of any material and in any form that will releasably attach to the mating surface of the opposing waist region when pressed against it. For example, the primary fastening component may be a mechanical fastener that releasably engages with the mating surface, such as by means of a plurality of hooks engaging with loops formed by fibers in a nonwoven sheet. Alternatively, the primary fastening component may be an adhesive that releasably adheres to the mating surface.

Still other variations are also possible. For example, the fasteners 72 may be disposed on the interior of the article 20 in the first waist region 40 such that the first waist region 40 overlaps the second waist region 42 when they are fastened together. As another example, the fasteners 70 may be disposed on the exterior of the article 20 rather than on the interior. As a further example, the fasteners 70 may be used with a specific mating fastener surface particularly suited for cooperation with the fasteners 70 (e.g., a loop layer that works with a hook fastener, or a layer particularly treated to provide a suitable contacting surface for a specific adhesive). Additionally exemplary fasteners and fastener arrangements, the fastening components forming these fasteners, and the materials that are suitable for forming fasteners are described in U.S. Published Application Nos. 2003/0060794 and 2005/0222546 and U.S. Pat. No. 6,428,526, among others.

It has been discovered during development of the present invention that the development of dressing and undressing skills as well as the development of continence are both related to and potentially important to a successful potty training experience. For example, a child may begin to recognize the urge to urinate and have an ability to control and delay the onset of urination. If such a child has a desire to use the toilet, but is wearing a disposable absorbent article which he or she does not have the dexterity to remove readily, the child may not be able to use the toilet successfully. Therefore, it may be desirable to provide “easy open” features, such as those described further below either with or without refastenability features in a garment in combination with the sensation members described herein to provide a garment having multiple potty training features in combination which synergistically re-enforce each other.

The disposable absorbent article 20 may be provided with an easy open feature such as shown in FIG. 2b. Such an easy open feature can comprise a single element or a combination of elements designed to make the seams of the article easier to open so as to remove the article. For example the easy open feature could comprise a gripping tab 74. Additionally an easy open bond line pattern 76 could be provided either alone or in combination with the gripping tab 74. The easy open bond line pattern 76 shown in FIG. 2b can act as a type of “zipper” structure allowing propagation of an opening force along the side of the article 20. Other easy open features could include a line of weakness, a notch or tab designed to propagate a tear, a tab gripping area or similar feature designed to allow for more easy release of a refastenable side fastener. Easy open features such as those described above can be provided if desired on articles having refastenable features or those which do not have refastenable fasteners—for example, a pant like garment with pre-formed side seams incorporating an easy open feature which can be used once, but which does not allow for refastenability once opened.

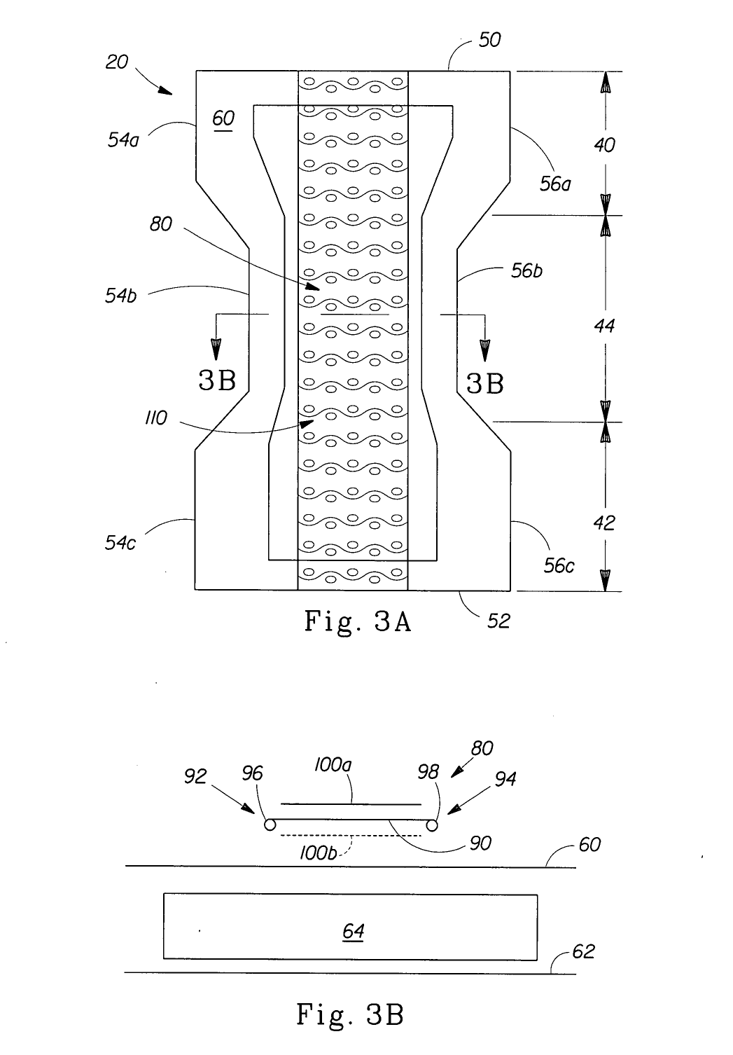

According to the present disclosure, the exemplary article 20, such as is illustrated in FIGS. 1 and 2, may be combined with or assembled to include a sensation member 80. The term sensation member is analogous to sensory element member and feedback response member as used herein or in copending applications having Attorney Docket 10352P, 10353P, 10355P and 10356P filed on Mar. 31, 2006. Several embodiments, some with one or more variations, are illustrated in FIGS. 3a, 3b, 4a, 4b, 5a, 5b, 6a, and 6b. Elements common to all embodiments are numbered similarly in all Figures, while those elements unique to each embodiment are numbered differently, with the sensation member according to a first embodiment being numbered as 80, a second embodiment as 180, and a third embodiment as 280. In addition, while the exemplary absorbent articles illustrated each include a single sensation member, the articles may include a plurality of sensation members according to other embodiments.

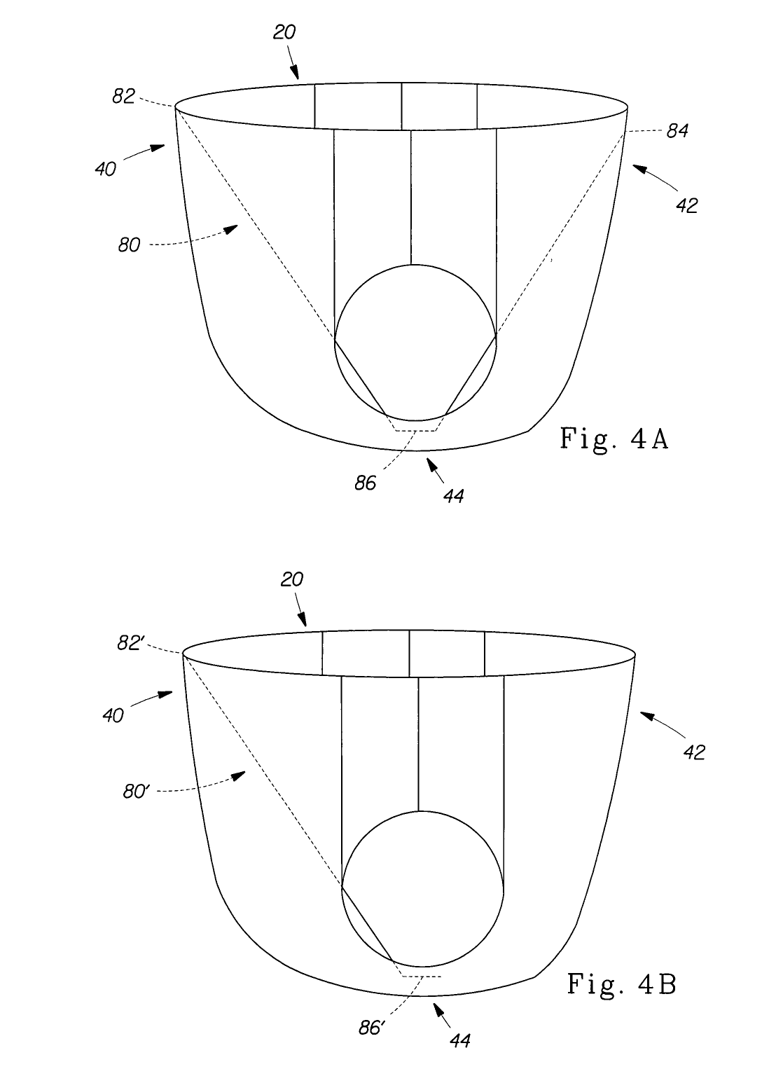

Turning then to the first embodiment of the exemplary sensation member 80, shown in FIGS. 3a, 3b, and 4a, the sensation member 80 illustrated is a structure that is formed separately from, but discretely attached to, the topsheet 60. In particular, and as seen best in FIG. 4a, the sensation member 80 has a first waist region 81, a first laterally extending joining region or end 82 attached to the first waist region 40, a second waist region 83 and a second longitudinally opposing and laterally extending joining region or end 84 attached to the second waist region 42. In addition, the sensation member 80 may have a center joining region 86 that may be attached to the crotch region 44. It is believed that the attachment of the member 80 to the crotch region 44 may assist in stabilizing the member 80, in facilitating fitting of the article 20 to the wearer, in preventing interference with bowel movements and in ensuring good contact with the wearer's skin.

As seen in FIG. 3b, the sensation member 80 may comprise a hydrophilic layer 90, alone or in combination, as explained in greater detail below. Exemplary materials suitable for use in the layer 90 include nonwovens, foams, woven materials, etc. In particular, the layer 90 may comprise, by way of illustration and not limitation, rayon, Lyocell and other cellulose-based materials, cotton, polyester, polypropylene and polypropylene blends (e.g., with other listed materials, such as a Lyocell/polypropylene blend), and hydrophilic forms of nonwovens such as SM (spunbond meltblown), SMS (spunbond meltblown spunbond), and SMMS (spunbond meltblown meltblown spunbond).

The layer 90 has first and second sides 92, 94 that may be parallel to the longitudinal axis 30 of the article 20. Preferably, but not necessarily, a first elastic member 96 may be attached to the layer 90 at the first side 92, while a second elastic member 98 may be attached to the layer 90 at the second side 94. The elastic members 96, 98 may extend along the entire length of the layer 90, or only a portion thereof. A fully or partially elasticized layer 90 may to tend to draw the sensation member 80 toward or against the skin of the wearer. Alternatively, the layer 90 may be formed to have a lesser length than another layer disposed relatively exteriorly, etc.

In one embodiment, the elastic members 96, 98 can be attached to the layer 90 such that at least the first waist portion 81 is foreshortened at a Contraction Ratio at a 10 g load of at least 0.1, preferably at least 0.15 and more preferably at least 0.2. One skilled in the art will understand that the first waist region 81 may be foreshortened by at least one elastic member by first applying a tension or stretching an elastic member, and applying the elastic member under tension against the layer 90. The elastic member under tension may then be adhesively attached to at least a portion of the layer 90. Once the adhesive has “dried”, the tension of the elastic member may be released thereby causing the portion of the layer 90 that is adhesively bonded to the elastic to contract. The degree of foreshortening of the layer 90 may be controlled by the amount of tension applied to the elastic member, as well as its thickness and/or diameter before it is adhesively attached to the layer as well as the location and length of the portion of the layer that is adhesively attached to the elastic member. In one embodiment, the second waist portion 83 may be foreshortened at a Contraction Ratio at a 10 g load of less than 0.2, preferably less than 0.15 and more preferably less than 0.1.

The Contraction Ratio at a 10 g load of the first and/or second foreshortened waist regions can be measured according to the following method.

The layer of the sensation member that includes the elastic member(s) (or an integral elastic formed by selective mechanical activation of a material that requires activation) is cut to form a test sample that includes the foreshortened portion.

One end of the sample including the elastic member(s) is fixedly attached via a clamp such that the other end is left hanging. A vertical gauge, that is placed along the sample, is marked at the level where the sample is clamped (i.e. top end portion of the sample where the elastic member and the layer are attached to each other).

A 10 g weight clip is then clipped to the other (i.e. hanging) end of the sample at the location where the adhesive was last applied (i.e. the lowest point where the elastic member is attached to the layer of the sensation member).

A 200 g weight is added to the clip and is supported by hand. The weight is then lowered gently such that the sample is subjected to the downward force of the 210 g clip and weight.

The vertical gauge that is placed along the sample is marked at the level of the clipped end of the sample (i.e. the end portion of the sample where the elastic member and the layer are attached to each other).

The 200 g weight is then removed and the sample is allowed to contracted gently (i.e. by raising the 10 g clip by hand) until it reaches it fully contracted state under a 10 g load.

After 10 seconds, another mark is made to the gauge at the level of the clipped end of the sample under a load of 10 g.

The two distances between the clamped portion and respectively the clipped end under a 10 g load and a 210 g load are recorded.

The Contraction Ratio is obtained by dividing the smallest distance (i.e. sample subjected to a 10 g load) by the longest distance (i.e. sample subjected to a 210 g load.

In one embodiment, the first waist region 81 is elastically stretchable from a relaxed (or fully contracted) state to a fully elongated state. In one embodiment, the force required to elongate a foreshortened region of the sensation member from a relaxed state to 85% of its length in a fully elongated state is at least 0.4 N, preferably at least 0.5 N, more preferably at least 0.6 N.

The force required to elongate a foreshortened region of the sensation member from a relaxed state to 85% of its length in a fully elongated state can be measured according to the following method.

The layer of the sensation member that includes the adhesively bonded or attached elastic member(s) is cut to form a test sample that includes the foreshortened portion. At least two substantially identical samples of a sensation member are prepared as discussed previously.

One end of a first simple is fixedly attached to a clamp and a load is applied to the other end until the sample starts breaking. The length of the “breaking” elongated sample is recorded and is referred to as its 100% or maximum length.

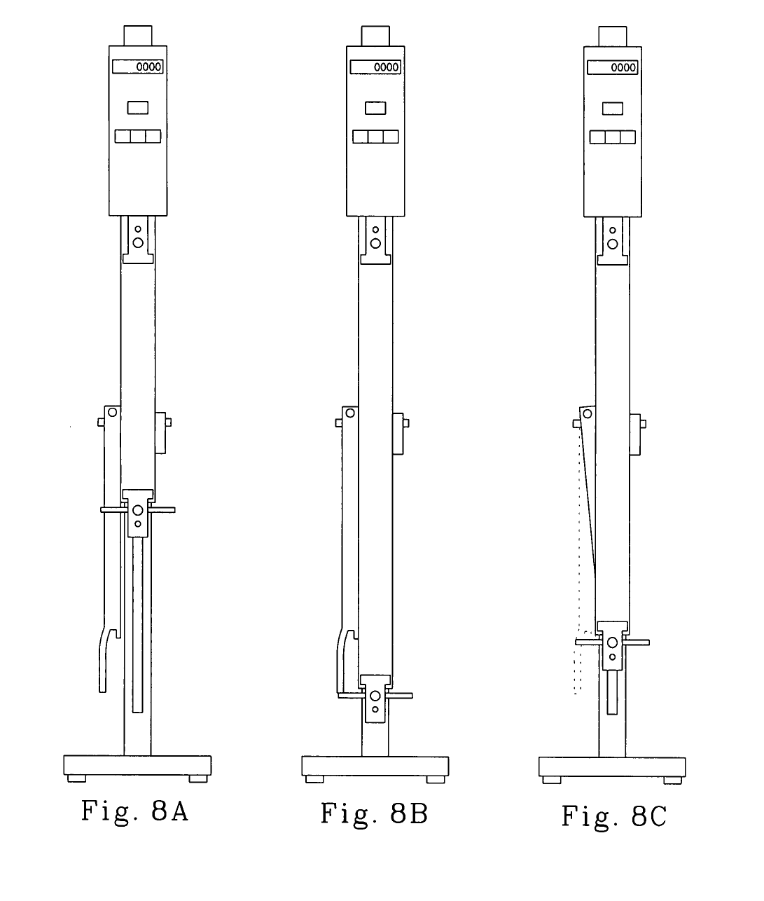

One end of the second sample (i.e. top end portion of the sample where the elastic member and the layer are attached to each other) including the elastic member(s) is fixedly attached via the upper clamp of a force stand available from Queen City Supply, 1859 Section Rd, Cincinnati, Ohio 45237 and the other end of the sample at the location where the adhesive was last applied (i.e. the lowest point where the elastic member is attached to the layer of the sensation member—or the lowest active point of elastication) is attached to the lower clamp of the device as schematically represented in FIG. 8a. The upper clamp is connected to a digital force gauge having a precision of 0.001 Newton.

The lower clamp of the device is lowered until the sample is elongated to 95% of its maximum length as schematically represented in FIG. 8b.

The lower clamp is then moved back up until the sample is elongated to 85% of its maximum length as schematically represented in FIG. 8c. When the sample reaches 85% of its maximum length, the force that is applied to the sample is then recorded and referred to as the force required to elongate a foreshortened region of the sample from a relaxed state to 85% of its length in a fully elongated state.

Without intending to be bound by any theory, it is believed that a sensation member having at least a foreshortened waist region as previously described and the Contraction Ratio at a 10 g load and/or elongation force previously discussed can remain in closer contact with the wearer's skin, in particular when the wearer is moving or in a standing position. Consequently, it is believed that the foreshortened region(s) of the sensation member enhance the effectiveness of the signal delivered to the wearer.

In one embodiment, the sensation member 80 consists essentially of the hydrophilic layer 90. That is, it may be sufficient for the purpose of alerting the wearer to an insult of urine that the urine be maintained for a period of time in the layer 90, thereby providing a sensation to the wearer. However, according to other variations of this embodiment, an active component, such as coating or agent, may be applied at 100a, 100b to the layer 90, which may be referred to as a support layer, as illustrated in solid and dashed line.

Thus, according to a second variation, the sensation member 80 may also comprise an active component in the form of a hydrophilic coating, which may be applied at 100a as shown in solid line in FIG. 3b. The hydrophilic coating may be disposed in a face-to-face arrangement with the support layer 90. Moreover, as illustrated, the hydrophilic coating may be disposed on the surface of the layer 90 closer to the wearer's skin (i.e., body-facing surface).

It will be also recognized that the hydrophilic coating may include a diverse range of materials, including lotions, creams and the like. Exemplary hydrophilic coatings include surfactants, such as the NUWET silicone surfactant available from GE Silicones of Wilton, Conn.

Further, according to a third variation, the sensation member 80 may also comprise an active component in the form of a temperature sensation agent (composition or structure), which may be applied at 100a. The temperature sensation agent may be disposed in face-to-face arrangement with the support layer 90. Moreover, as illustrated, the temperature sensation agent may be disposed on the surface of the layer 90 closer to the wearer's skin (i.e., body-facing surface). Further, the temperature sensation agent may be disposed on the support layer 90 in place of the hydrophilic coating, in conjunction with the hydrophilic coating, or combined (e.g., mixed) with the hydrophilic coating.

In one embodiment, the sensation member 80 may be at least a portion of the topsheet 60 that is impregnated with a material, for example at the raised central region 90 of the topsheet 60 between the elastic members 96 and 98. Accordingly, the sensation member 80 can be drawn upwards with the central region 90 towards or against the skin of the wearer. In one embodiment, the topsheet 60 can be divided into one or more impregnation zones bound as indicated by hidden lines. As illustrated, the impregnation zones can be laterally aligned or longitudinally aligned. Furthermore, they can be co-planar with respect to the x-y plane, or could be non-coplanar, for instance depending upon the mode of impregnation and the material that is impregnated or coated (as is described below, the present invention contemplates auxiliary layers that can include the sensation member 80). While each zone includes a sensation material as illustrated, it should be appreciated that an individual zone or zones can be dedicated to one or more predetermined sensation materials, or not include a sensation material. Furthermore, the impregnation zones can be shaped, sized, and positioned as desired to produce the desired wetness sensation response to insults of urine. It should be further appreciated that impregnation can occur in more than one mode. For instance, a surface electron microscopic image of an exemplary topsheet 60 illustrates one or more layers of a plurality of fibers that are overlap to form interstices therebetween. In one mode, a given zone of the topsheet 60 can be impregnated by coating individual interior fibers (as opposed to merely the surface fibers) with a given sensation member material, thus maintaining the interstices between adjacent fibers. In a second mode, a given zone of the topsheet 60 can be impregnated both by coating the individual fibers and by filling the interstices with the sensation material. In still another mode, the interstices 70 can be filled with a given sensation member material without substantially coating the individual fibers.

The topsheet 60 can be partially impregnated with the sensation member material meaning that in a given impregnation zone, only a portion of the total interstitial volume is occupied by the material and/or only a portion of the total surface area of the fibers in the region is coated with the material. Alternatively, the topsheet 60 can be substantially impregnated, meaning that in a given region of topsheet impregnation, substantially all of the total interstitial volume is substantially filled with the material and/or substantially all of the fibrous surface area is substantially completely coated with the material in the desired topsheet region. It should thus be understood that, unless otherwise specified, the term “impregnated,” “impregnation,” or other variations of the word “impregnate” as used herein refer to any one of the modes of impregnation disclosed above, alone or in combination, unless otherwise specified. It should further be appreciated that the term “sensation material” as used herein includes a single sensation member material present alone or in combination with other sensation member materials. If more than one material is used in combination, it should be appreciated that the materials can be mixed, and the mixture can impregnate the topsheet 60, or that the materials can be impregnated at different zones 66 of the topsheet 60, or that the materials can be impregnated in the same zone of the topsheet 60 but at different locations throughout the thickness of the topsheet 60. Furthermore, the materials can become impregnated using different modes of impregnation.

In one embodiment, the sensation material is impregnated into the surface of topsheet 60 closer to the wearer's skin (i.e., body-facing surface), though it can be appreciated that the temperature sensation agent can be impregnated in any manner described above, and can be disposed anywhere between, and including, the body-facing surface and the opposing garment-facing surface of the topsheet 60 so long as the impregnated zone of the topsheet 60 influences the wetness profile of the topsheet 60.

It will be recognized that the temperature sensation agent may include those materials that produce a temperature change (i.e., involve an endothermic or an exothermic reaction), as well as those that produce the sensation that a temperature change has occurred without actually producing a temperature change. For example, the temperature sensation agent may be a cooling agent. Further, the cooling agent may be the AQUACOOL dye manufactured by United Polymer Technology of Akron, Ohio. The AQUACOOL dye is a water-soluble dye that changes temperatures when brought into contact with water. Another example of cooling agent may be menthol or a menthol derivative, which chemicals are believed to provide the sensation of a temperature change, while not actually producing a temperature change. The COOLACT P and COOLACT 10 products manufactured by LIPO Chemicals of Paterson, N.J. are examples of menthol derivative products which may be suitable. Other examples of temperature change agents (e.g., endothermic salts) that may be suitable temperature sensation agents may be found in U.S. Pat. No. 6,642,427.

Moreover, according to a fourth variation, the sensation member 80 may comprise an active component in the form of a hydrophobic coating, which may be applied at 100b as shown in dashed line in FIG. 3b. According to this variation, the hydrophilic coating and/or temperature sensation agent may or may not be included (the temperature sensation agent being combinable with either the hydrophilic or hydrophobic agent, if present). Like the hydrophilic coating, the hydrophobic coating may be disposed in a face-to-face arrangement with the support layer 90. Moreover, as illustrated, the hydrophobic coating may be disposed on the surface of the layer 90 between the layer 90 and the topsheet 60, or the surface further from the wearer's skin (i.e., the opposite surface).

It will be also recognized that the coating 100b may include a diverse range of materials, including lotions, creams etc. Exemplary coatings may comprise hydrophobic coatings (HFC) and liquid-impermeable surface coatings (LISC). In particular, the coating may be made in accordance with the disclosure of U.S. Published Application No. 2005/0177123. Alternatively, the coatings may be acrylic polymer (e.g., acrylamide, ethyl alcohol, n-butyl alcohol, methyl-methacrylate, acrylamide, acrylonitrile, or combinations thereof) emulsions manufactured and sold, for example, under the ROHATOL tradename by Lanxess Corp. of Pittsburg, Pa., the RH-MW1845K tradename by Rohm & Haas of Philadelphia, Pa., or the FA1, FA2, or FA3 tradenames by PolymerLatex International GmbH of Marl, Germany.

In fact, the hydrophilic and hydrophobic coatings and temperature sensation agents described above may be used with other sensation members, as will be discussed below. Moreover, the coatings and agents may be useful in conjunction with the structures described in U.S. Pat. No. 6,627,786, among others.

The spacing of the first and second sides 92, 94 of the layer 90 and the width of the coating, if present, may be determined to allow enough liquid to bypass the sensation member 80 to the core 64 so as to prevent flooding. Flooding may result in leakage of the article 20 during urination, which is undesirable in the article 20 when it is a diaper or training pant, for example. Consequently, it will be recognized that the dimensions of the layer 90 and coating may be determined to prevent flooding while at the same time wicking sufficient liquid to create a sensation of wetness for the user.

During insults of urine, the layer 90 allows urine to penetrate in the z-direction and also provides a medium for the flow of urine in the x-y plane via wicking. The layer 90 and/or the coating may enhance the movement of the passage of the urine in the x-y plane, thereby expanding the wetted area of the sensation member, which preferably is held in contact with the wearer's skin. The wicking in the x-y plane causes the urine to spread out and effectively wet a large area before being absorbed into the absorbent assembly, thereby maximizing the wetness signal experienced by the wearer.

In fact, the sensation member 80 may have a high initial wetness that dries out after, for example, approximately 10 minutes. That is, while the initial wetness may vary whether the layer 90 is used alone, or in combination with a hydrophilic or hydrophobic coating, the preferred response would be for the initial wetness to be sufficient to cause the wearer to recognize the condition, and the wetness over time to be limited so as not to create, for example, skin health issues because of too much wetness being present near the skin over a prolonged period of time.

The outer cover 62 of the absorbent article, may include a substantially vapor permeable material. The outer cover 62 is generally constructed to be permeable to at least water vapor and has a moisture vapor transmission rate of at least 1000 g/m²/24 hr, preferably at least 2000 g/m²/24 hr, more preferably at least 3000 g/m²/24 hr., even more preferably at least 3500 g/m²/24 hr, most preferably at least 4000 g/m²/24 hr and even most preferably at least 4500 g/m²/24 hr. For example, the outer cover 62 may define a moisture vapor transmission rate of from 1000 to 10000 g/m²/24 hr. Materials which have a moisture vapor transmission rate less than those above do not allow a sufficient amount of air exchange and undesirably result in increased levels of humidity of the air inside the article during use.

Moisture Vapor Transmission Test

The Moisture Vapor Transmission Rate is measuring the amount of moisture adsorbed by Calcium-Chloride in a “cup” like container covered with the test specimen from controlled outside air conditions (40.+−0.3.degree. C./75.+−0.3% relative humidity).

The sample holding a cup is a cylinder with an inner diameter of 30 mm and an inside height from bottom to top flange of 49 mm. A flange having a circular opening to match the opening of the cylinder can be fixed by screws, and a silicone rubber sealing ring, matching the inner diameter, fits between the top flange and the cylinder. The test specimen is to be positioned such that it covers the cylinder opening, and can be tightly fixed between the silicone rubber sealing and the upper flange of the cylinder.

The equipment as well as the test specimen should be well adjusted to the temperatures, and the constant temperature/humidity chamber preferably has a size to accommodate up to 30 samples.

The absorbent desiccant material is CaCl2, such as can be purchased from Wako Pure Chemical Industries Ltd., Richmond, Va., US under the product designation 030-00525. If kept in a sealed bottle, it can be used directly. It also can be sieved to remove lumps, or excessive amounts of fines, if existing. It also can be dried at 200° C. for about 4 hrs. 15.0.+−0.0.02 g of CaCl2. are weighed into the cup, and tapped lightly so as to level it out, such that the surface is about 1 cm from the top of the cup.

The samples, which are cut to about 3.2 cm by 6.25 cm, are placed flat and overlapping with the seal over the opening, and the seal and the top flange are affixed by the screws without over tightening. The total weight of the cup assembly is accurately recorded on a four decimal places: scale, and the assembly is placed into the constant temperature/humidity chamber.

After 5 hrs (without opening of the chamber), the sample is removed and immediately covered tightly with non-vapor permeable plastic film such as Saran wrap as commonly used in the U.S. After about 30 mins to allow for temperature equilibration, the plastic film cover is removed and the accurate weight of the assembly is recorded.

The MVTR value is then calculated from the moisture increase during these 5 hours through the 3 cm circular opening and then converted to units of “g/24 h/m²”.

For each test, three replicates should be run, the resulting values will be averaged, and the result rounded to the nearest 100 value.

Overall, this method is applicable to thin films, multi layer laminates and the like. Experience has shown, that typical standard deviations range between 50 and 250 g/24 hr/m² for averaged values of up to about 5000 g/24 hr/m².

Due to this range, materials being considered to be essentially vapor impermeable such as conventional PE films, are reported as having a MVTR of about 200 g/24 hr/m².

If the units for an MVTR value are omitted for simplicity, a material “having a MVTR value of 1000” should accurately be a material “having a MVTR value of 1000 g/24 h/m²” according to this method.

The outer cover 62 is also desirably substantially liquid impermeable. For example, the outer cover may be constructed to provide a hydrohead value of at least 60 cm, desirably at least 80 cm, and more desirably at least 100 cm when subjected to the Hydrostatic Pressure Test. Materials which have hydrohead values less than those above undesirably result in the strike through of liquids, such as urine, during use. Such fluid strike through can undesirably result in a damp, clammy feeling on the outer cover 62 during use.

Hydrostatic Pressure Test

The Hydrostatic Pressure Test is a measure of the liquid barrier properties of a material. In general, the Hydrostatic Pressure Test determines the height of water (in centimeters) in a column which the material will support before a predetermined amount of water passes through. A material with a higher hydrohead value indicates it is a greater barrier to liquid penetration than a material having a lower hydrohead value. The Hydrostatic Pressure Test is performed according to Method 5514—Federal Test Methods Standard No. 191A.

The outer cover 62 may be composed of any suitable materials which either directly provides the above desired levels of liquid impermeability and air permeability or, in the alternative, materials which can be modified or treated in some manner to provide such levels. In one embodiment, the outer cover 62 may be a nonwoven fibrous web constructed to provide the required level of liquid impermeability. For example, a nonwoven web composed of spunbonded or meltblown polymer fibers may be selectively treated with a water repellent coating or laminated with a liquid impermeable, vapor permeable polymer film to provide the outer cover 62. In a particular embodiment of the invention, the outer cover 62 may comprise a nonwoven web composed of a plurality of randomly deposited hydrophobic thermoplastic meltblown fibers which are sufficiently bonded or otherwise connected to one another to provide a substantially vapor permeable and substantially liquid impermeable web. The outer cover 62 may also comprise a vapor permeable nonwoven layer which has been partially coated or otherwise configured to provide liquid impermeability in selected areas.

In one embodiment, the absorbent article may exhibit increased breathability when wet in order to lower the level of relative humidity of the air inside the article in particular after the first insult of urine when compared to conventional absorbent articles. One skilled in the art will understand that when a sensation member that is “wetted” by a first insult of urine and is thereafter in contact with the wearer's skin, some of the urine may ultimately flow inside the absorbent core, but some of the urine that is retained in the sensation member may evaporate from the garment facing surface of the sensation member and/or the wearer's skin. This evaporation of urine causes a local drop in temperature that may be perceived by the wearer. This local drop of temperature may be of at least 2° C., preferably at least 5° C., more preferably at least 10° C. It may be beneficial to maintain the level of relative humidity of the air inside the diaper to maintain a good evaporation rate so as to intensify and/or lengthen the coolness sensation perceived by the wearer. It has been discovered that the ability of the absorbent articles of the present invention to exhibit a reduced level of relative humidity of the air inside the article during use depends, at least in part, on the ability of the absorbent article to achieve a high rate of air exchange within the article. Moreover, it has been further discovered that the achievement of such reduced levels further depends on the ability of the article to maintain or increase the high rate of air exchange when wet.

The ability of an absorbent article to achieve high rates of air exchange when dry and/or when wet has, for the purposes of this application, been quantified as the Dry Air Exchange Rate, the Wet Air Exchange Rate and the Wet Air Exchange Rate/Dry Air Exchange Rate ratio as determined according to the Tracer Gas Test set forth below. Briefly, the Tracer Gas Test involves injecting a tracer gas at a constant rate inside the absorbent article next to the skin of the wearer while the article is being worn. Simultaneously, the concentration of the tracer gas in the air space between the article and the wearer is measured by withdrawing a sample at the same constant rate as the injection. The air exchange is then determined based on mass balances of the tracer gas and the air within the space in question.

To achieve the desired reduced levels of relative humidity, the absorbent articles of the different aspects of the present invention may be constructed to define a Wet Air Exchange Rate of at least 190 cubic centimeters per minute, preferably at least 200 cubic centimeters per minute, more preferably at least 225 cubic centimeters per minute, even more preferably at least 250 cubic centimeters per minute, and most preferably at least 300 cubic centimeters per minute. For example, the absorbent articles may define a Wet Air Exchange Rate of from 175 to 1500 cubic centimeters per minute and preferably from about 225 to 1500 cubic centimeters per minute. Absorbent articles which exhibit Wet Air Exchange Rates less than those above do not allow a sufficient amount of air exchange and undesirably result in increased levels of relative humidity of the: air inside the article during use, which in turn, lowers the evaporation rate causing the coolness sensation.

The absorbent articles of the different aspects of the present invention may be further constructed to define a Wet Air Exchange Rate/Dry Air Exchange Rate ratio of at least 0.20, preferably at least 0.23, more preferably at least 0.27, and most preferably at least 0.30 for improved performance of the sensation member. In one embodiment, the absorbent article may be constructed to define a Wet Air Exchange Rate/Dry Air Exchange Rate ratio of at least 0.50, preferably at least 0.70, more preferably at least 0.80, and most preferably at least 0.90 for improved performance. In one embodiment, the absorbent article is constructed to define a Wet Air Exchange Rate/Dry Air Exchange Rate ratio of at least 1.00, preferably at least 1.05, and more preferably at least 1.10 for improved performance of the sensation member. For example, the absorbent articles may define a Wet Air Exchange Rate/Dry Air Exchange Rate ratio of from 0.20 to 2.00, preferably from about 0.50 to 2.00, more preferably from about 0.70 to 2.00, and even more preferably from about 1.00 to 2.00 for improved performance of the sensation member. Absorbent articles which define such high Wet Air Exchange Rate/Dry Air Exchange Rate ratios provide the improved air exchange when it is needed the most, i.e. when the article is wetted in order to deliver a strong signal to the wearer via the sensation member.

In one embodiment, the absorbent article may further be constructed to define a Dry Air Exchange Rate of at least 525 cubic centimeters per minute, preferably at least 575 cubic centimeters per minute, more preferably at least 625 cubic centimeters per minute, even more preferably at least 675 cubic centimeters per minute, and most preferably at least 750 cubic centimeters per minute for improved performance of the sensation member. For example, the absorbent articles may define a Dry Air Exchange Rate of from 525 to 2500 cubic centimeters per minute and preferably from about 575 to 2500 cubic centimeters per minute. Absorbent articles which exhibit Dry Air Exchange Rates less than those above do not allow a sufficient amount of air exchange and undesirably result in increased levels of relative humidity of the air inside :the diaper during use (in particular after the first insult or subsequent insults of urine).

Alternatively, in one embodiment wherein the absorbent article defines a relatively high Wet Air Exchange Rate/Dry Air Exchange Rate ratio it may not be necessary for such article to provide Dry Air Exchange Rates set forth above to achieve improved performance. For example, in embodiments wherein the absorbent article defines a Wet Air Exchange Rate/Dry Air Exchange Rate ratio of at least 0.50, preferably at least 0.70, more preferably at least 0.80 and even more preferably at least 0.90, the absorbent article may define a Dry Air Exchange Rate of at least 200, preferably at least 225, more preferably at least 250 and even more preferably at least 300 cubic centimeters per minute for improved performance of the sensation member.

The improved air exchange within the absorbent article may lead to a more intense and/or longer drop of the skin temperature of the wearer in use.

Tracer Gas Test

The Tracer Gas Test is a measure of the rate of air exchange in garments such as absorbent articles and is a steady flow/steady state test described generally in TAPPI JOURNAL., Volume 80, No. 9, September 1997. In general, the air exchange rate values are calculated from the measured mass exchange within the garment. The test involves injecting a tracer gas at a constant rate inside the article next to the outer surface of the torso of a mannequin while the article is secured about the mannequin. Simultaneously, the concentration of the tracer gas in the air space between the article and the mannequin is measured by withdrawing a sample at the same constant rate as the injection. The air exchange rate is then being determined based on mass balances of the tracer gas and the air within the space in question. The Tracer Gas Test is completed as follows:

Equipment

1. Mannequin—The test is conducted with Step 3 or Step 4 sized diapers designed for infants weighing from about 16 to 28 pounds and from about 22 to 37 pounds, respectively. The articles are placed on mannequins which have the following dimensions:

Mannequin 1

| | |
|---|---|
| height (waist to knees) | 26 centimeters |
| circumference at waist | 42 centimeters |
| circumference at hips | 44 centimeters |
| thigh circumference | 22 centimeters |

Mannequin 2

| | |
|---|---|
| height (waist to knees) | 28 centimeters |
| circumference at waist | 48 centimeters |
| circumference at hips | 51 centimeters |
| thigh circumference | 27 centimeters |

2. A test area which is environmentally controlled to 20° C. and 50% relative humidity.

3. CO₂ Analyzer—An infrared CO₂ Analyzer such as Model 17515A commercially available from Vacu-Med Vacumetrics, 4483 McGrath Street #102, Ventura, Calif.

4. Rotameters—Rotameters to maintain gas flow rates such as Matheson Rotameter Model TS-35 commercially available from Specialty Gases Southeast Inc., 3496 Peachtree Parkway, Suwanee, Ga.

5. Gas Cylinders—Two gas cylinders of calibrated medical grade gas at a pressure of 4 kPa from Specialty Gases Southeast Inc., 3496 Peachtree Parkway, Suwanee, Ga. The tracer gas includes 5% CO₂ and air and the calibration gas is 100% air.

Procedure

1. Turn the CO₂ analyzer on. After it has been on for 30 minutes, calibrate the analyzer with the calibration gas and adjust the flow control to achieve a flow rate of 150 cubic centimeters per minute through the analyzer.

2. Place the diaper to be tested on the mannequin.

3. Turn on the CO₂ tracer gas flow. The flow rate of the injected tracer gas into the space between the diaper and the mannequin must be equal to the sample flow rate through the CO₂ analyzer (150 cc/min.).

4. Measure and record the concentration (C) of the tracer gas (CO₂) in the air space between the diaper and the mannequin every 10 seconds for 20 minutes. The data over the last 10 minutes are averaged and used to calculate the air exchange rate as follows:

Air Exchange Rate=150 cc/min* [(C_{T}−C)/(C−C₀)]

wherein,

C_{T}=concentration of the tracer gas (5%)

C=concentration of the tracer gas in the space being measured

C_{O} concentration of the tracer gas in the chamber environment (0.04%)

The Dry Air Exchange Rate is the air exchange rate as determined according to the above procedure before the diaper has been subjected to any insults. The Wet Air Exchange Rate is the air exchange rate determined according to the above procedure except that once the diaper is secured to the mannequin, 180 milliliters (Step 3) or 210 milliliters (Step 4) of adjusted 0.9 weight percent aqueous saline is added in three insults of 60 or 70 milliliters each at a rate of 15 milliliters/second with a 45 second delay between insults. The Wet Air Exchange Rate/Dry Air Exchange Rate ratio is determined by dividing the Wet Air Exchange Rate by the Dry Air Exchange Rate for the same sample.

As previously discussed, the effectiveness of the signal delivered to the wearer by the sensation member may be reduced if the portion of the sensation member which is supposed to be in contact with the wearer's skin is pulled away from the wearer's skin prematurely.

Without intending to be bound by any theory, it is believed that the effectiveness of the sensation member can be increased by the careful selection of material(s) used to make at least portion of the sensation member capable of contacting the wearer's skin such that this portion has certain properties.

It is believed that the portion of the sensation member, which delivers a signal to the wearer, should not only be in contact with his or her skin but remain thereto for a prolonged period of time.

The inventors have found that the ability of a material to remain in contact with the wearer's skin during use can be quantified via at least one parameter or a combination of parameters chosen from at least one the surface area, the Stiffness of the material, the Skin Contact Time of the material and the Wet Peel Factor of the material.

When a portion of the sensation member is insulted with urine and this wetted portion gets in contact with the wearer's skin, it is believed that its ability to effectively deliver a signal to the wearer depends at least partially on the ability of the wetted portion to conform to shape of the wearer's anatomy. One skilled in the art will understand that if the portion of the sensation member that is capable of contacting the wearer's skin, is made of a stiffer or less flexible material, this portion will not conform as readily to complex shapes (i.e. other than flat) and, as a result, the sensation member may not be able to signal the presence of urine to the wearer as effectively.

In one embodiment, at least a portion of the sensation member has a Stiffness of less than 100 N/m, preferably less than 75 N/m, more preferably less than 50 N/m and most preferably less than 25 N/m in either the Machine direction and/or the Cross machine direction of said portion of the sensation member. The inventors have found that the Stiffness of a material may be lowered when the material is wetted.

In one embodiment, the portion of the sensation member having the previously discussed Stiffness has a surface area of between 10 cm² and 500 cm², preferably between 25 cm² and 500 cm² and more preferably 100 cm² and 400 cm².

A suitable method to measure the Stiffness of a portion of a sensation member is as follows.

Stiffness and Bending Force

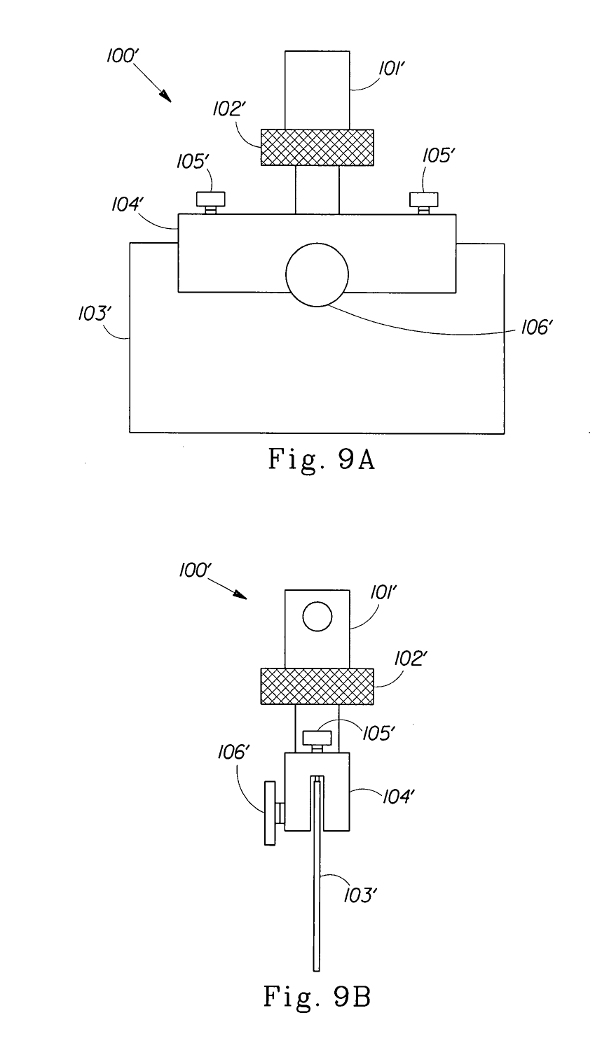

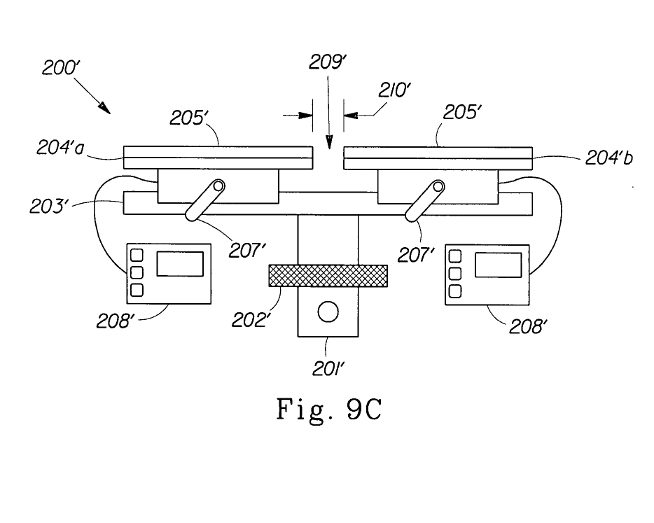

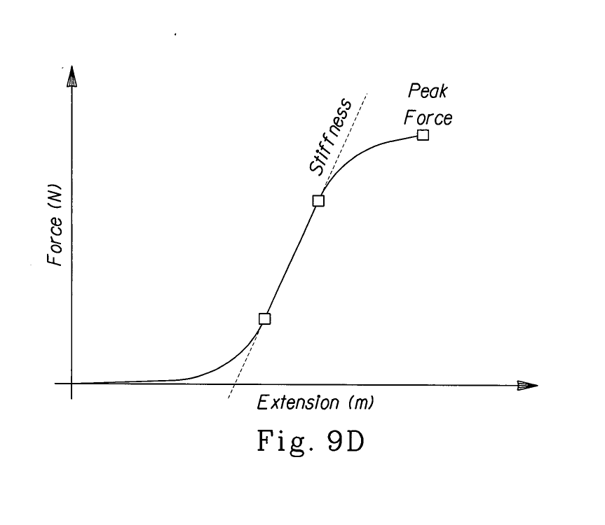

Stiffness of a sample is measured using a constant rate of extension tensile tester with computer interface (a suitable instrument is a MTS Alliance under TestWorks 4 software, as available from MTS Systems Corp., Eden Prairie, Minn.) fitted with a 10 N load cell. A plunger blade 100′ shown in FIG. 9a (side view) and FIG. 9b (front view) is used for the upper movable test fixture and base support platform 200′ shown in FIG. 9c are used as the lower stationary test fixture. All testing is performed in a conditioned room maintained at 23° C.±2° C. and 50%±2% relative humidity.

Components of the plunger 100′ are made of a light weight material to maximize the available load cell capacity. The shaft 101′ is machined to fit the tensile tester and has a locking collar 102′ to stabilize the plunger and maintain alignment orthogonal to base support platforms 200′. The blade 103′, is 115 mm long by 65 high by 3.25 mm wide, and has a material contact edge with a continuous radius. The bracket 104 is fitted with set screws 105′ that are used to level the blade and a main set screw 106′ to firmly hold it in place after adjustment.

The bottom fixture 200′ is attached to the tensile tester with the shaft 201′ and locking collar 202′. Two movable support platforms 204′ are mounted on a rail 203′. Each test surface 205′ is 85 mm wide by 115 mm long and has a minimal coefficient of friction. Each platform has a digital position monitor 208′ which reads the individual platform positions, and set screws 207′ to lock their position after adjustment. The two platforms 204′ are square at the gap edge and the plate edges should be parallel front to back. The two platforms form a gap 209′ with an adjustable gap width 210′.

Accurately (±0.02 mm) align the plunger blade 103′ so that it is orthogonal to the top surface of the support platforms 204′ and exhibits no skew relative to their gap edges. Using the position monitors 208′, accurately set the gap 210′ to 8.00±0.02 mm between the two gap edges of the support platforms 204′, with the plunger blade 103′ accurately (+0.02 mm) centered in the gap. Program the tensile tester for a compression test. Set the gage length from the bottom of the plunger blade 103′ to the top surface of the support platform 204′ to 15 mm. Set the crosshead to lower at 500 mm/min for a distance of 25 mm. Set the data acquisition rate to 100 Hz.

Precondition samples at 23° C.±2° C. and 50%±2% relative humidity for 2 hours prior to testing. Die cut a test specimen 25.4 mm by 25.4 mm. Place the specimen flat onto the surface of the support platform 204′ over the gap 209′, Center the specimen across the gap, its edges parallel and perpendicular to the gap. Zero the load cell; start the tensile tester and the data acquisition.

Program the software to calculate the maximum peak force (N) and stiffness ( N/m) from the constructed force (N) verses extension (m) curve. Stiffness is calculated as the slope of the force/extension curve for the linear region of the curve (see FIG. 9d). A minimum line segment of at least 25% of the peak force should be used to calculate the slope.

It is also believed that when a portion of the sensation member is insulted with urine and this wetted portion gets in contact with the wearer's skin, its ability to effectively deliver a signal to the wearer also depends at least partially on the ability of the wetted portion to remain in contact with the wearer's skin for a prolonged period of time. One skilled in the art will understand that if the portion of the sensation member that is capable of contacting the wearer's skin, is pulled away from the wearer's skin too quickly, the signal may be too short to provide effective awareness of the presence of body exudates to the wearer.

The inventors have found that in order to provide an effective signal to the wearer, at least a portion of the sensation member has a Skin Contact Time of at least 30 sec, preferably at least 60 sec, more preferably at least 300 sec and most preferably at least 600 sec.

In one embodiment, the portion of the sensation member having the previously discussed Skin Contact Time has a surface area 10 cm² and 500 cm², preferably between 25 cm² and 500 cm² and more preferably 100 cm² and 400 cm².

A suitable method for determining the Skin Contact Time of a material under wet conditions is as follows:

“Open” an absorbent article (diaper) and remove the sensation member.

Cut a sample of about 10 cm² (for example about 3.2 cm×3.2 cm) from the sensation member.

The sample needs to be taken from the portion of the sensation member that is capable of contacting the skin of the wearer. It shall be understood that the sample may also include other components of the sensation member that are not necessarily part of the layer of the sensation member that is capable of contacting the skin of the wearer but that contributes to the delivery of a signal to the wearer. For example, a sample of a sensation member comprising a body contacting layer and a patch of material (including a sensation agent) attached to and located underneath the garment facing surface of the body contacting layer, will also include the patch of material. However, it will be also understood that a sample does not include other components of the article that are not contributing to the delivery of a sensation to the wearer (for example, the absorbent core that may include a fluid acquisition component, a fluid distribution component, and/or a fluid storage component. An example of a suitable absorbent core having a fluid acquisition component, a fluid distribution component, and a fluid storage component is described in U.S. Pat. No. 6,590,136 to Young et al., issued Jul. 8, 2003 and assigned to The Procter & Gamble Company.).

The sample is soaked into a 0.9% saline heated to 37° C.±1° C. for at least 2 minutes to saturate the sample. All testing is performed in a conditioned room maintained at 23° C.±2° C. and 50%±2% relative humidity.

The sample is removed and left to “drip” for 30 sec in order to remove excess liquid.

The sample is placed flute against a vertical, flat and smooth stainless steel plate.

Once the sample is placed on the stainless steel plate, a stop watch is used to measure the time the sample remains attached to the surface.

The stop watch is stopped when at least a portion of the sample starts being detached from the stainless steel plate and the Skin Contact Time is recorded.

It is also believed that when a portion of the sensation member is insulted with urine and this wetted portion gets in contact with the wearer's skin, its ability to effectively deliver a signal to the wearer also depends at least partially on the ability of the wetted portion to remain in contact with the wearer's skin while being subjected to a peeling force. One skilled in the art will understand that a wearer will rarely stay still for a prolonged period of time. Movements of the wearer, in particular in the lower torso or abdomen region of the wearer may cause the sensation member to be pulled away from the wearer's skin prematurely and, as a result, the signal intended to be delivered by sensation member may be too short to provide effective awareness of the presence of body exudates to the wearer.

The inventors have found that the Wet Peel Force of at least a portion of a sensation member may impact the effectiveness of the sensation delivered to the wearer's skin.

In one embodiment, at least a portion of a sensation member has a Wet Peel Force of at least 30 grams force, preferably at least 40 grams force, more preferably at least 50 grams force and even more preferably at least 75 grams force. The inventors have found that when at least a portion of a sensation member is wetted and has a Wet Peel Force of at least 30 grams force, said wetted portion is capable of remaining against the skin of the wearer in particular when the wearer is moving. As a result, the sensation (for example a temperature change sensation member) may be more effectively delivered to the wearer's skin. A non-limiting example of a suitable material having a Wet Peel Force of at least 30 grams force may be a nonwoven material having a contact promoting substance that adheres gently to the wearer's skin and resists casual disengagement that is disposed (via coating or impregnation) on at least a portion of the skin contacting surface of the nonwoven material. Non-limiting examples of contact promoting substances may include skin care compositions, for instance lotions as described in U.S. Pat. Nos. 5,607,760; 5,609,587; 5,635,191; 5,643,588; 5,968,025; 6,118,041; 6,120,488; 6,120,783; 6,153,209; 6,156,024; and 6,166,285, sticky lotions as described in WO 2004/087092, waxes, tacky polymers and adhesives such as body adhesives. In certain embodiments a water-activatable adhesive may be desirable as it would only promote contact once the wearer urinates. Water activatable adhesives for use in disposable absorbent articles are disclosed in U.S. Pat. No. 6,623,465, which is incorporated by reference. The skin contact promoting substance may be disposed on at least a portion of the sensation member 80 or in a region of the topsheet 60 or other supporting structure in proximity to the sensation member 80.

In one embodiment, at least a portion of a sensation member has a Wet Peel Force of less than 30 grams force, preferably less than 25 grams force, more preferably less than 20 grams force, even more preferably less than 15 grams force. The inventors have found that when at least a wetted portion of a sensation member has a Wet Peel Force of less than 30 grams force, said wetted portion is capable of being “peeled” or removed from the skin of the wearer in particular when the wearer is moving until further movement of the wearer causes said portion to come in contact with the wearer's skin. The inventors have found that when such a wetted portion of a sensation member is periodically in contact and then removed from the wearer's skin, the succession of contacts and removals may cause discomfort which provides an effective signal to the wearer of the insult of urine. As a result, the sensation (in particular a wetness sensation) may be more effectively delivered to the wearer's skin. A non-limiting example of a suitable material having a Wet Peel Force of less than 30 grams force may be a hydrophobic or a hydrophobic nonwoven material having a basis weight of less than 50 gsm.

In one embodiment, a sensation member may have at least a first portion having a Wet Peel Force of at least 30 grams force, preferably at least 40 grams force, more preferably at least 50 grams force and even more preferably at least 75 grams force, and at least a second portion having a Wet Peel Force of less than 30 grams force, preferably less than 25 grams force, more preferably less than 20 grams force, even more preferably less than 15 grams force. A non-limiting example of a material having a first portion and a second portion has previously described may be a nonwoven material including a contact promoting substance that is applied (via coating or impregnation) in discrete regions separated by at least one region which doest not include a contact promoting substance. The contact promoting substance may be applied in a continuous pattern or a discrete pattern.

In one embodiment, the portion of the sensation member having the previously discussed Wet Peel Force has a surface area 10 cm² and 500 cm², preferably between 25 cm² and 500 cm² and more preferably 100 cm² and 400 cm².

A suitable method for determining the Wet Peel Force of a material under wet conditions is as follows:

### 90 Degree Wet Peel Force

The 90 Degree Wet Peel Force is measured using a constant rate of extension tensile tester with computer interface (a suitable instrument is a MTS Alliance under TestWorks 4 software, as available from MTS Systems Corp., Eden Prairie, Minn.) fitted with a 10N load cell. The bottom stationary test fixture used is described in ASTM D 6862-04, “Standard Test Method for 90 Degree Peel Resistance of Adhesive.” The upper movable fixture is a pneumatic jaw, fitted with rubber faced grips wider than the width of the test specimen. The specimen is tested against stainless steel with a grind surface of 320 granulation. The gage between the upper grips and the bottom fixture is set to 25 mm.

Cut a test sample of 25.4 mm wide (transverse direction of the garment) by 100 mm long (longitudinal direction of the garment) from the sensation member of an absorbent article. It shall be understood that the sample may also include other components of the sensation member that are not necessarily part of the layer of the sensation member that is capable of contacting the skin of the wearer but that contributes to the delivery of a signal to the wearer. For example, a sample of a sensation member comprising a body contacting layer and a patch of material (including a sensation agent) attached to and located underneath the garment facing surface of the body contacting layer, will also include the patch of material. However, it will be also understood that a sample does not include other components of the article that are not contributing to the delivery of a sensation to the wearer (for example, the absorbent core that may include a fluid acquisition component, a fluid distribution component, and/or a fluid storage component. An example of a suitable absorbent core having a fluid acquisition component, a fluid distribution component, and a fluid storage component is described in U.S. Pat. No. 6,590,136 to Young et al., issued Jul. 8, 2003 and assigned to The Procter & Gamble Company.).

Immerse the sample in 0.9% saline heated to 37° C.±1° C. for 2 minutes to saturate, then remove and suspend vertically for 30 sec. to drain excess fluid. Lay the specimen, without applying pressure, onto the surface of the steel test surface with the surface of interest (typically the surface which is capable of contacting the skin of a wearer) facing the stainless steel plate. The sample should be in intimate contact with the surface, free from air bubbles trapped under the sample. Peel approximately 35 mm of the sample strip up from the test surface and place 10 mm into the upper grip. Align the sample and test fixture such that the sample end in the upper grips hangs perpendicular to the test surface, and the sample forms a 90 degree angle at the point of contact with the test surface. When a test sample is too stiff to form readily a 90 degree angle, gently bend approximately 35 mm of the sample in order to form a 90 degree angle. The vertical portion of the sample should be taunt but not exerting more than 0.05 N on the load cell.

Start the tensile tester and data collection. The upper fixture is moved away from the bottom fixture at a crosshead speed of 300 mm/minute for 60 mm. Simultaneously, the test surface of the bottom fixture is moved horizontally at the same rate as the cross head to maintain a 90 degree peel throughout the experiment. The average peel force (N) between an extension of 25 mm and 50 mm is calculated by the software from the recorded force/extension curve.

The inventors have found that it may be sufficient for the purpose of alerting the wearer to an insult of urine that a sufficient surface moisture quantity of urine be maintained for a period of time in the layer 90, thereby providing a wetness sensation to the wearer. In one embodiment, the Wetness Density (as measured by the Wetness Density Test, below) of at least a portion of the sensation member may range from about 0.4 to about 1.5 grams at 60 seconds.

Wetness Density Test

Purpose

This test simulates the introduction of urine into a training pant diaper. No pressure is applied while loading to simulate the baby urinating in a standing position.

Equipment

| | |
|---|---|
| Template | Flat Base unit on which to mount the test product |
| Filter Paper | Ahlstrom Filtration Paper Code 632, 127 × 127 mm |
| Balance | accuracy +/−0.01 g |
| Saline | 0.9% Saline heated to 37° C. +/− 1° C. |
| Graduated Cylinder | Convenient Source |
| Timer | Convenient Source, time measurements taken to |
| | nearest second |
| Weight | Plastic Dimensitons/Weight 127 × 127 mm, |
| | 297 grams |
| Metal Cylinder | Metal Cylinder, Inside Diameter = 60 mm, |
| | Outside Diameter = 70 mm, Thickness of |
| | ring = 10 mm, Height = 40 mm, |
| | Weight = 327 grams. |

Test Procedure
1. Test fluid is 0.9% saline heated to 37° C.±1° C.
2. If the product is provided in a closed, pant-like form, open side seams of all products. If product contains defined side seams product should be opened at those locations.
Otherwise, cut side panels with scissors at midpoint of side panels.
3. Mount the test product with clamps onto a flat template in a flat stretched out condition to ensure no wrinkles in the topsheet or liners. The pant elastics should stay intact.
4. Weigh one piece of filter paper.
5. Measure as follows to define the loading point (boy) 10.2 cm below front edge of the core, or (girl) 12.7 cm below front edge of the core. (unisex or generic) use boy measurement
6. Measure 75 mls of 0.9 saline (heated to 37° C.±1° C.) into the graduated cylinder.
7. Center the cylinder over the loading point and pour the saline from the graduated cylinder at the loading point. Loading should be done over approximately 5 seconds or at approximately 15 mL/second.
8. Once fluid is poured, start timer. Pouring the saline is considered to be the “urination event” for purposes of comparison of time with time parameters in the claims.
9. After 60 seconds have elapsed, place filter paper on the topsheet and then the plastic weight to ensure complete contact between the filter paper and the topsheet. The weight should be lowered slowly and applied gently to the filter paper.
10. After 10 seconds from weight application, lift the weight and filter paper off of the topsheet and weigh the filter paper.
11. Calculate wet weight minus dry filter paper weight in mg. This value is divided by 161.29 cm² to determine the wetness density in mg/cm².

The inventors have found that gender specific absorbent articles, each having a sensation member, may include different properties in order to better deliver the signal delivered by the sensation member depending on the gender of the wearer. A non-limiting example of such an array can be a first absorbent article associated and/or designed for a female wearer and a second absorbent article associated and/or designed for a female wearer. One skilled in the art will understand it can be beneficial to adjust the properties of an absorbent article having a sensation member based on anatomical differences, as well as, behavioral differences between a male wearer and a female wearer.

The inventors have also found that absorbent articles having different sizes, each having a sensation member, may include different properties in order to better deliver the signal delivered by the sensation member depending on the development stage of the wearer. A non-limiting example of such an array can be a first absorbent article associated and/or designed for a first stage of development of a wearer and a second absorbent article associated and/or designed for a second stage of development of a wearer. One skilled in the art will understand it can be beneficial to adjust the properties of an absorbent article having a sensation member based on size of the wearer.

In one embodiment, a first absorbent article having a first sensation member having at least one property chosen from at least one of surface area, Contraction Ratio at a 1 g load, force required to elongate a foreshortened region of the sensation member from a relaxed state to 85% of its length in a fully elongated state, Stiffness, Skin Contact Time, Wet Peel Force, can be associated with a second absorbent article having a second sensation member having at least one property chosen from at least one of Contraction Ratio at a 10 g load, force required to elongate a foreshortened region of the sensation member from a relaxed state to 85% of its length in a fully elongated state, Stiffness, Skin Contact Time, Wet Peel Force such that at least one of the property(ies) of the second absorbent article is different (i.e. has a different value than) from the corresponding property of the first absorbent article. The first and second absorbent articles can be associated to form an array of absorbent articles. Among other benefits, an array of absorbent articles as previously discussed allow a caregiver or any other person making the purchase decision to select the absorbent article that will better fit a wearer depending on the gender and/or development stage of the wearer.

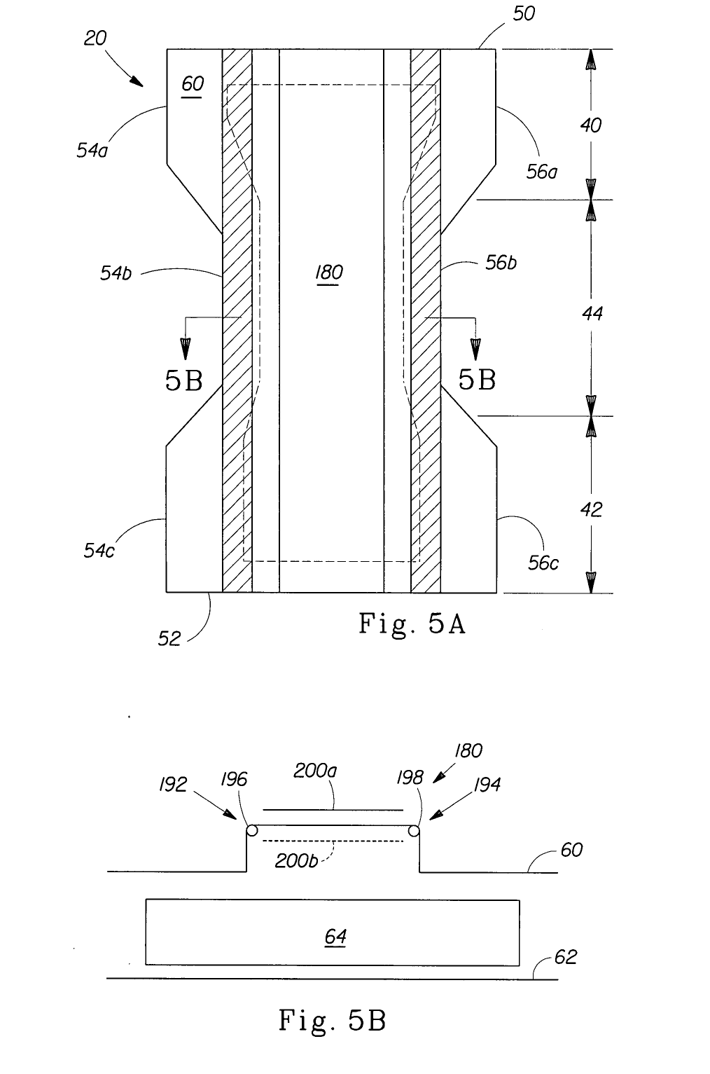

Turning next to FIGS. 5a and 5b, a second embodiment of a sensation member 180 is illustrated therein. Similar to the sensation member 80, the sensation member 180 has first and second sides 192, 194 that are arranged parallel to the longitudinal axis 30 of the article 20. Moreover, elastic members 196, 198 may be attached to the sensation member 180 at the sides 192, 194 so as to elasticize the sensation member 180, which may assist in bringing the sensation member 180 in to close contact with the skin of the wearer. Further, the sensation member may include an active component, such as (i) a coating, which may be a hydrophilic coating disposed on a body-facing surface or a hydrophobic coating disposed on an opposite surface, (ii) a temperature sensation agent, which may be disposed on either surface and in substitution for, in conjunction with, or combined with the coating, and/or (iii) a layer of hydrophilic material, such as was described relative to the layer 90 above and which also may be disposed on a body-facing surface.

The sensation member 180 differs from the sensation member 80 in that the structure corresponding to the support layer 90 is formed from a section of the topsheet 60 spaced from the core 64. That is, a section of the topsheet 60 is folded to define support layer structure of the member 180, and, in particular, is folded along the sides 192, 194. The elastic members 196, 198 are then disposed beneath the topsheet 60 in the space between the topsheet 60 and the core 64. In this fashion, the sensation member 180 may be integrated to a greater degree to the remainder of the article 20 than the member 80, thereby reducing the likelihood that the sensation member 180 will become detached from the remainder of the article 20.

The sensation member 180 may include other features in common with the sensation member 80. For example, the coatings and agents disposed at 200a, 200b may include those exemplary coatings and agents listed above. Moreover, while not illustrated, a visible graphic 110 may be include on a surface of the sensation member 180, providing one or more of the advantages discussed above.

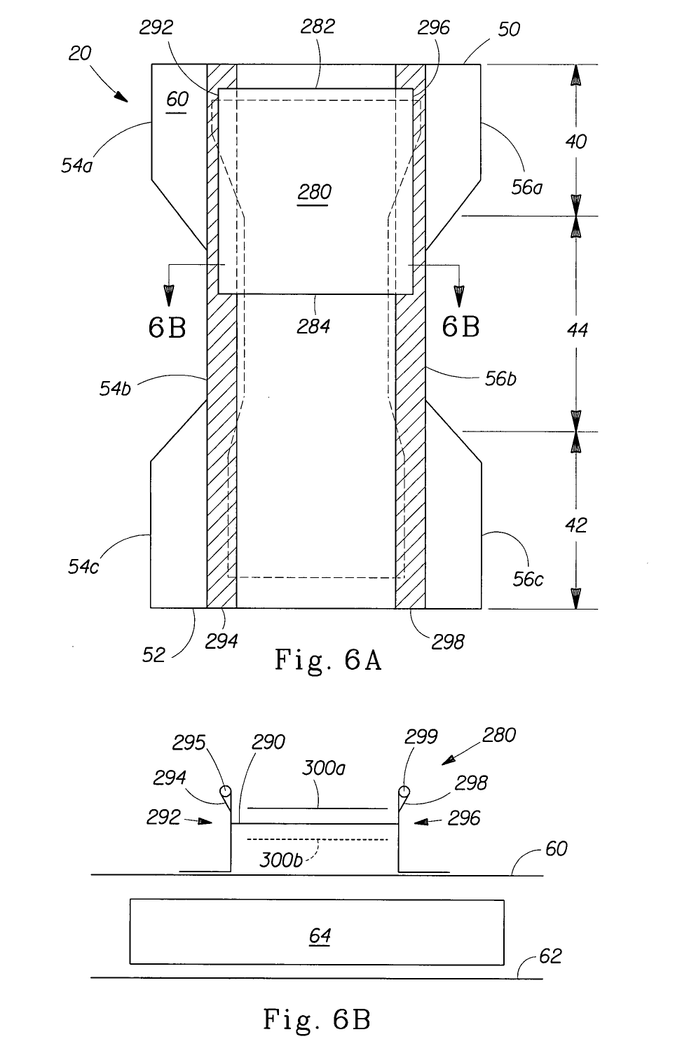

Turning then to FIGS. 6a and 6b, a third embodiment of the sensation member 280 is illustrated, with the barrier leg cuffs folded back slightly in FIG. 6a to expose the sensation member 280. The sensation member 280 has a first laterally extending end 282 and a second longitudinally opposing and laterally extending end 284. As will be recognized, the distance between the ends 282, 284 is shorter than the distance between the ends 50, 52, or even the distance between end 50 and the crotch region 44. According to the embodiment, the position of the ends 282, 284 relative to the ends 50, 52 and the spacing between the ends 282, 284 is such that the likelihood that the sensation member 280 will be wetted with urine is enhanced.

The sensation member 280 includes a layer 290. The layer 290 extends between the ends 282, 284. Additionally, a first longitudinal edge 292 of the layer 290 is attached to a first barrier leg cuff 294 attached to the topsheet 60, while a laterally opposed, longitudinal edge 296 is attached to a second, spaced barrier leg cuff 298, also attached to the topsheet 60. Moreover, each barrier leg cuff 294, 298 includes an elastic member 295, 299. In this fashion, it is not necessary to attach separate elastic members to the support layer 290, but the elastic members 295, 299 of the barrier leg cuffs 294, 298 instead may urge the sensation member 280 into contact with the skin of the wearer.

In fact, it is believed that the attachment of the sensation member 280 to the barrier leg cuffs 294, 298 may permit greater control over the spacing of the sensation member 280 relative to the topsheet 60 (i.e., distance between member 280 and topsheet 60) than had heretofore been possible. That is, by attaching the sensation member 280 along its sides 292, 296, rather than at its ends 282, 284, the spacing of the member 280 relative to the topsheet 60 may be better controlled than in those embodiments wherein the member is attached at its ends, or potentially even in those embodiments where the member is integrated into the topsheet 60 and elastic members disposed internal to the topsheet 60 are used to define, at least in part, the sensation member. Additionally, by attaching the sensation member 280 to the leg cuffs 294, 298, the dimension of the sensation member 280 perpendicular to the longitudinal axis may be greater than, for example, the sensation members 80, 180 discussed above.

Similar to the embodiment shown in FIGS. 3a and 3b, the sensation member 280 may consist essentially of the layer 290, or may comprise the layer 290 in combination with a coating or an agent, which coating or agent may be disposed in a face-to-face arrangement with the layer 290, which may be referred to as a support layer. The coating may be a hydrophilic coating disposed at 300a on the body-facing surface of the layer 290, or a hydrophobic coating disposed at 300b on the opposite surface. Additionally, a temperature sensation agent may be disposed at 300a on either surface and in substitution for, in conjunction with, or combined with the coating.

The sensation member 280 may include other features in common with the sensation member 80, 180. For example, the coatings and agents may include those exemplary coatings and agents listed above. Additionally, while not illustrated, a visible graphic 110 may be include on a surface of the sensation member 280, providing one or more of the advantages discussed above.

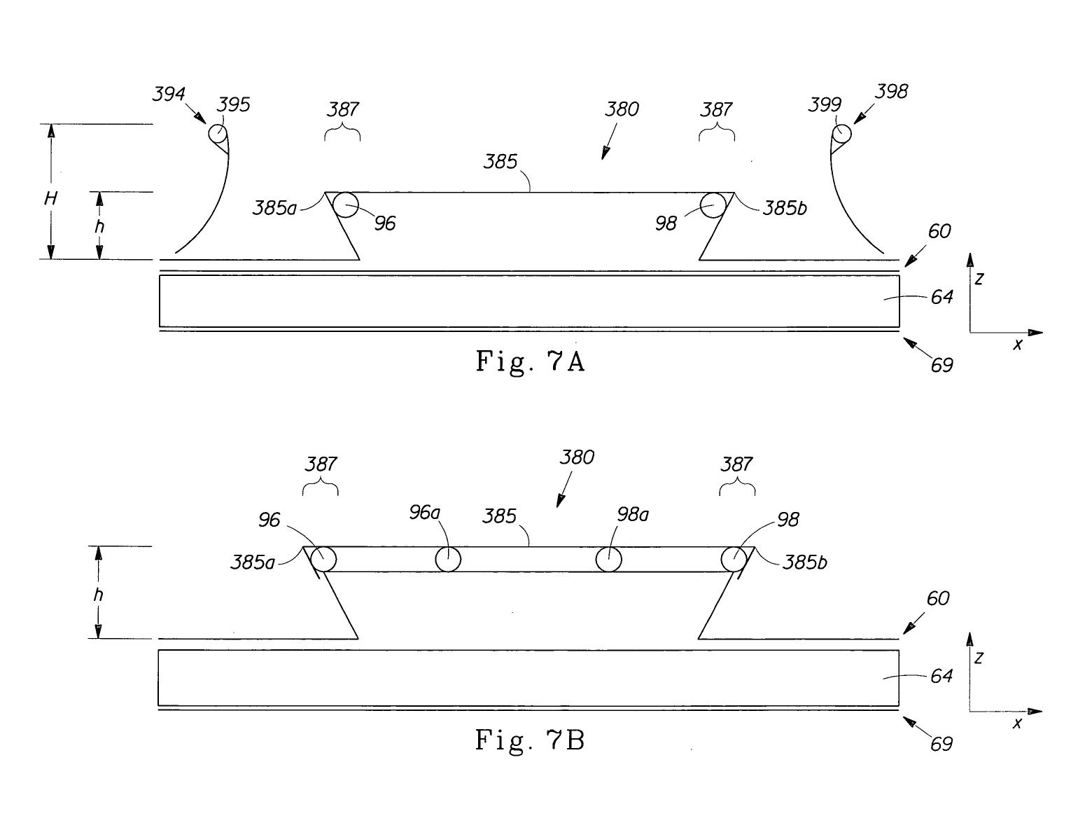

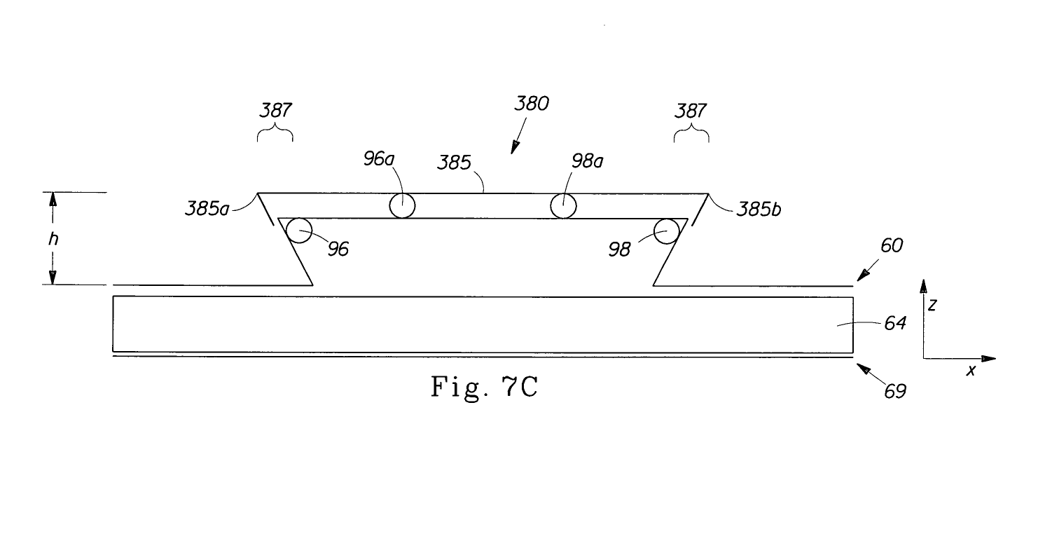

In additional embodiments shown in FIGS. 7A-7C, the sensation member 380 or any layer including the sensation member can be disposed in two parallel Z-folds 387 formed along the longitudinal length of the absorbent article. The Z-folded sensation member 380 or any layer including the sensation member may be attached to the underlying layers along the longitudinal edges of the topsheet 60 allowing the portion between the Z-folds of the topsheet 60 to float freely. Elastic elements 396, 397 may be disposed along the central region of the sensation member 380 in order to deflect the central region outward away from the absorbent core 64. Elastic elements 396, 397 may be disposed between layers of the topsheet 60, between layers of the sensation member 380, between the topsheet 60 and sensation member 380, or any other configuration that connects the elastic elements 396, 397 to the topsheet 60 and/or sensation member 380. The central region 385 may have a first side edge 385a and a second side edge 385b such that at least one of said side edges 385a, 385b has a projected height h measured the z direction between the side edge and the base of the sensation member that connects the sensation member to the absorbent article.

A disposable absorbent article including a sensation member is attached to the inner surface of a curved plate (i.e. the concave surface relative to the hypothetical center of the circle having the same curvature as the plate) having a radius of curvature of about 250 mm. The disposable absorbent article is attached to the plate such that its garment facing surface (i.e. outer cover) is in contact with the plate. In this configuration, the elastic member(s) that are disposed longitudinally on the disposable absorbent article are in an elongated configuration and are applying a force that is pulling any layer attached to the elastic member away from the core. A ruler having one end contacting the base of the sensation member and the other end pointing toward the center of the hypothetical circle formed by the curved plate, may be used to measure the distance between the base of the sensation and the side edge of the sensation member.

The Z-folded sensation member 380 allows the central region 385 to be suspended away from the core 64 and the topsheet 60. The combination of the Z-folded sensation member 380 and the elastic elements 396, 397 maintains the sensation members in proximity to the wearer's skin in the event that the diaper sags or fits loosely around the wearer.

Alternatively, additional elastic elements 396a, 397a may be disposed along the central region of the Z-folded sensation member. Elastic elements 396a, 397a, may be disposed between layers of topsheet 60, between layers of the sensation member 380, between the topsheet 60 and sensation member 380, or any other configuration that connects the elastic elements 396a, 397a to the topsheet 60 and/or sensation member 380. Elastic elements 396a, 397a provide additional support to prevent sagging and promote contact with the wearer's skin.

The absorbent article may also include a first barrier leg cuff 394 and a second barrier leg cuff 398, which may include elastic members 395, 399 respectively. First and second barrier leg cuffs are disposed on the absorbent article such that the Z-folded sensation member 380 is located between the barrier leg cuffs 395, 399. At least one of the first barrier leg cuff 394 and a second barrier leg cuff 398 has a projected height H measured the z direction between an upper edge of the barrier leg cuff and the base of the barrier leg cuff that connects the barrier leg cuff to the absorbent article.

The projected height h and H may be measured according the following method.

A disposable absorbent article including a sensation member is attached to the inner surface of a curved plate (i.e. the concave surface relative to the hypothetical center of the circle having the same curvature as the plate) having a radius of curvature of about 250 mm. The disposable absorbent article is attached to the plate such that its garment facing surface (i.e. outer cover) is in contact with the plate. In this configuration, the elastic member(s) that are disposed longitudinally on the disposable absorbent article are in an elongated configuration and are applying a force that is pulling any layer attached to the elastic member away from the core. A ruler having one end contacting the base of the sensation member and the other end pointing toward the center of the hypothetical circle formed by the curved plate, may be used to measure the distance between the base of the sensation and the side edge of the sensation member. The side edge of the sensation member is gently extended to its maximum height (i.e. without applying a force that would cause the sensation member to be torn or destroyed) and then record the measurement. The projected height measurement can be repeated at various points along the sensation member in order to determine its maximum projected height. The ruler may be moved such that one end is in contact with the base of an outer leg cuff and its other end is pointing towards the center of the hypothetical circle passing through the curved plate. The projected height H may be determined by measuring distance between the base of the outer leg cuff and the upper edge of the outer leg cuff. The upper edge of the outer leg cuff is gently extended to its maximum height (i.e. without applying a force that would cause the outer leg cuff to be torn or destroyed) and then record the measurement. The projected height measurement can be repeated at various points along the outer leg cuff in order to determine its maximum projected height.

In one embodiment, the projected height h of at least one of the first side edge 385a and a second side edge 385b is between 90% and 300%, preferably between 100% and 250%, more preferably between 100% and 200% of the projected height H of at least one of the first barrier leg cuff 394 and a second barrier leg cuff 398.

In one embodiment, the projected height h of at least one of the first side edge 385a and a second side edge 385b is between 15 mm and 50 mm, preferably between 20 mm and 45 mm, more preferably between 25 mm and 40 mm.

In addition to incorporating the wetness sensation member with the topsheet, the wetness sensation member of the present invention may also be integrated with other components of the diaper: such as the barrier leg cuffs. The barrier leg cuffs may be made from either permeable or impermeable material. In either case, the barrier leg cuff material may form one of the layers of the wetness sensation member.

In addition to the features described above, the disposable absorbent article 20 may also include a variety of features known in the art, such as slit openings, outer leg cuffs, front and rear ear panels, waist cap features, elastics, and the like to provide desired fit, containment, and aesthetic characteristics. Such additional features are well known in the art and are described in U.S. Pat. Nos. 3,860,003, 5,151,092, and 6,482,191 among others. Additionally, a transfer layer, which may also be referred to as an acquisition or distribution layer, may be disposed between the topsheet 60 and the core 64. Moreover, the elements discussed above may be modified from their illustrated forms.

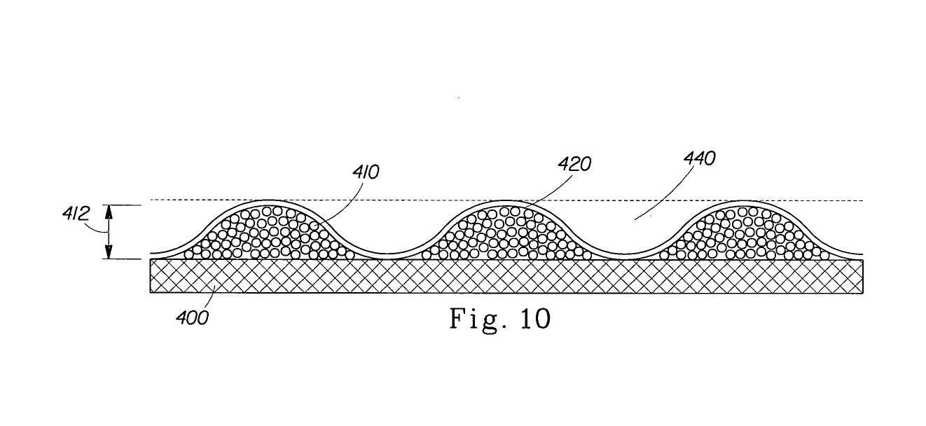

One preferred embodiment of the present invention includes, but is not limited to, articles described in U.S. Patent Application No. 2004/0162536 and U.S. Patent Application No. 2004/0167486. The aforementioned applications are directed to absorbent articles having an absorbent core which imparts increased wearing comfort to the article and makes it thin and dry. As shown in FIG. 10, the absorbent articles of the present invention may comprise an absorbent core 64 comprising a substrate layer 400, absorbent polymer material 410 and a fibrous layer of adhesive 420. The substrate layer 400 is preferably provided from a non-woven material, preferred non-wovens include those provided from synthetic fibers, such as PE, PET and PP. As the polymers used for non-woven production are inherently hydrophobic, they are preferably coated with hydrophilic coatings.

In accordance with the present invention, the absorbent material is immobilized when wet such that the absorbent core achieves a wet immobilization of more than 50%, preferably of more than 60%, 70%, 80% or 90%.

The substrate layer 400 comprises a first surface and a second surface. At least portions of the first surface of the substrate layer 400 are in direct contact with a layer of absorbent polymer material 410. This layer of absorbent polymer material 410 is preferably a discontinuous layer, and comprises a first surface and a second surface. As used herein, a discontinuous layer is a layer comprising openings. Typically, these openings have a diameter or largest span of less than 10 mm, preferably less than 5 mm, 3 mm, 2 mm and a span of more than 0.5 mm, 1 mm or 1.5 mm. At least portions of the second surface of the absorbent polymer material layer 410 are in contact with at least portions of the first surface of the substrate layer material 400. The first surface of the absorbent polymer material 410 defines a certain height 412 of the layer of absorbent polymer above the first surface of the layer of substrate material 400. When the absorbent polymer material layer 410 is provided as a discontinuous layer, portions of the first surface of the substrate layer 400 are not covered by absorbent polymer material 410. The absorbent core 64 further comprises a thermoplastic composition 420. This thermoplastic composition 420 serves to at least partially immobilize the absorbent polymer material 410.

In one preferred embodiment of the present invention the thermoplastic composition 420 can be disposed essentially uniformly within the polymeric absorbent material 410.

However, in an even more preferred embodiment of the present invention the thermoplastic material 420 is provided as a fibrous layer which is partially in contact with the absorbent polymer material 410 and partially in contact with the substrate layer 400. In this preferred structure the absorbent polymer material layer 410 is provided as a discontinuous layer, a layer of fibrous thermoplastic material 420 is laid down onto the layer of absorbent polymeric material 410, such that the thermoplastic layer 420 is in direct contact with the first surface of the layer of absorbent polymer material 410, but also in direct contact with the first surface of the substrate layer 400, where the substrate layer is not covered by the absorbent polymeric material 410. This imparts an essentially three-dimensional structure to the fibrous layer of thermoplastic material 420 which in itself is essentially a two-dimensional structure of relatively small thickness (in z-direction), as compared to the extension in x- and y-direction. In other words, the fibrous thermoplastic material layer 420 undulates between the first surface of the absorbent polymer material 410 and the first surface of the substrate layer 400.

Thereby, the thermoplastic material 420 provides cavities to hold the absorbent polymer material 410, and thereby immobilizes this material. In a further aspect, the thermoplastic material 420 bonds to the substrate 400 and thus affixes the absorbent polymer material 410 to the substrate 400. Highly preferred thermoplastic materials will also penetrate into both the absorbent polymer material 410 and the substrate layer 400, thus providing for further immobilization and affixation.

Of course, while the thermoplastic materials disclosed herein provide a much improved wet immobilization (i.e., immobilization of absorbent material when the article is wet or at least partially loaded), these thermoplastic materials also provide a very good immobilization of absorbent material when the article is dry.

In accordance with the present invention, the absorbent polymer material 410 may also be mixed with absorbent fibrous material, such as airfelt material, which can provide a matrix for further immobilization of the super-absorbent polymer material. However, preferably a relatively low amount of fibrous cellulose material is used, preferably less than 40 weight %, 20 weight %, or 10 weight % of cellulose fibrous material as compared to the weight of absorbent polymer material 410. Substantially airfelt free cores are preferred. As used herein, the term “absorbent fibrous material” is not meant to refer to any thermoplastic material 420 even if such thermoplastic material is fiberized and partially absorbent.

The absorbent core of the present invention may further comprise a cover layer. This cover layer may be provided of the same material as the substrate layer 400, or may be provided from a different material. Preferred materials for the cover layer are the non-woven materials. In this embodiment, portions of the cover layer bond to portions of the substrate layer 400 via the thermoplastic material 420. Thereby, the substrate layer 400 together with the cover layer provides cavities to immobilize the absorbent polymer material 410.

The areas of direct contact between the thermoplastic material 420 and the substrate material 400 are referred to as areas of junction 440. The shape, number, and disposition of the areas of junction 440 will influence the immobilization of the absorbent polymer material 410. The areas of junction can be of squared, rectangular, or circular shape. Preferred areas of junction are of circular shape. Preferably, they have a diameter of more than 0.5 mm, or 1 mm, or 1.5 mm and of less than 10 mm, or 5 mm, or 3 mm, or 2 mm. If the areas of junction 440 are not of circular shape, they preferably are of a size as to fit inside a circle of any of the preferred diameters given above.

The areas of junction 440 can be disposed in a regular or irregular pattern. For example, the areas of junction 440 may be disposed along lines. These lines may be aligned with the longitudinal axis of the absorbent core, or alternatively, they may have a certain angle in respect to the longitudinal edges of the core. It has been found, that a disposition along lines parallel with the longitudinal edges of the absorbent core 64 create channels in the longitudinal direction which lead to a lesser wet immobilization. Preferably, therefore the areas of junction 440 are arranged along lines which form an angle of 20 degree, 30 degree, 40 degree, or 45 degree with the longitudinal edges of the absorbent core 64. Another preferred pattern for the areas of junction 440 is a pattern comprising polygons, for example pentagons and hexagons or a combination of pentagons and hexagons. Also preferred are irregular patterns of areas of junction 440, which also have been found to give a good wet immobilization.

Two fundamentally different patterns of areas of junctions 440 can be chosen in accordance with the present invention. In one embodiment, the areas of junctions are discrete. They are positioned within the areas of absorbent material, like islands in a sea. The areas of absorbent materials are then referred to as connected areas. In an alternative embodiment, the areas of junctions can be connected. Then, the absorbent material can be deposited in a discrete pattern, or in other words the absorbent material represents islands in a sea of thermoplastic material 420. Hence, a discontinuous layer of absorbent polymer material 410 may comprise connected areas of absorbent polymer material 410 or may comprise discrete areas of absorbent polymer material 410.

In a further aspect of the present invention, it has been found that absorbent cores providing for a good wet immobilization can be formed by combining two layers. In this embodiment, the absorbent core material comprises two substrate layers 400, two layers of absorbent polymer material 410 and two layers of fibrous thermoplastic materials 420. When two discontinuous layers of an absorbent polymer material 410 are used, they would be typically arranged in such a way that the absorbent polymer material of the one layer faces the areas of junction 440 of the other layer. In an alternative preferred embodiment, however, the areas of junction 440 are offset and do not face each other.

According to the present invention, the thermoplastic layer 420 can comprise any thermoplastic composition, preferred are adhesive thermoplastic compositions, also referred to as hot melt adhesives. A variety of thermoplastic compositions are suitable to immobilize absorbent material. Some initially thermoplastic materials may later lose their thermoplasticity due to a curing step, e.g., initiated via heat, UV radiation, electron beam exposure or moisture or other means of curing, leading to the irreversible formation of a crosslinked network of covalent bonds. Those materials having lost their initial thermoplastic behaviour are herein also understood as thermoplastic materials 420.

In embodiments of the present disclosure, a disposable wearable absorbent article can include a stretchable outer cover. For example, the outer cover can be a uniaxially stretchable outer cover, configured to stretch in one direction. Also as an example, the outer cover can be a biaxially stretchable outer cover, configured to stretch in two directions. In various embodiments, the outer cover can be configured as described in US non-provisional patent application entitled “Biaxially Stretchable Outer Cover for an Absorbent Article,” filed on Nov. 15, 2006 with Express Mail No. EV916939625 and further identified by attorney docket number 10643, which is hereby incorporated by reference.

In embodiments of the present disclosure, a disposable wearable absorbent can include an outer cover configured in various ways, including configurations of part or all of the outer cover as stretchable, non-stretchable, with an elastic nonwoven, with an elastic film and extensible nonwoven, with an extensible film and an elastic nonwoven, pre-stretched with elastic strands allowed to contract, mechanically activated, with zero strain laminate, and/or combinations of these and any other outer cover configurations. In various embodiments of the present disclosure, a disposable wearable absorbent article can include a printed outer cover with various basis weights, chemistries, and/or mechanical activations, as will be understood by one of ordinary skill in the art.

The term sensation member is analogous to sensory element member and feedback response member as used herein or in copending applications Attorney Docket 10352M, 10353M, 10354M, 10355M and 10356M filed on Mar. 31, 2007.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A refastenable absorbent article having a waist region and a crotch region, the article comprising:
an outer cover, wherein said outer cover has a moisture vapor transmission rate of at least 1000 g/m²/24 hr;
a sensation member having a body-facing surface, wherein at least a portion of the body facing surface of said sensation member is capable of contacting the skin of a wearer and wherein said portion has a Stiffness of less than about 100 N/m; and
an absorbent core disposed between the outer cover and the sensation member.

2. The absorbent article of claim 1 wherein said portion has a Stiffness of less than about 75 N/m.

3. The absorbent article of claim 2 wherein said portion has an area of at least 10 cm².

4. The absorbent article of claim 3 wherein said sensation member includes a waist portion wherein said waist portion is foreshortened at a Contraction Ratio at a 10 g load of less than about 0.2.

5. The absorbent article of claim 4 wherein said foreshortened waist portion is stretchable from a relaxed state to a fully elongated state and wherein the force required to elongate said foreshortened region of said sensation member from a relaxed state to 85% of its length in a fully elongated state is at least 0.4 N.

6. A refastenable absorbent article having a waist region and a crotch region, the article comprising:
an outer cover, wherein said outer cover has a moisture vapor transmission rate of at least 1000 g/m²/24 hr;
a sensation member having a body-facing surface, wherein at least a portion of the body facing surface of said sensation member is capable of contacting the skin of a wearer and wherein said portion has a Skin Contact Time of at least 30 seconds; and
an absorbent core disposed between the outer cover and the sensation member.

7. The absorbent article of claim 6 wherein said portion has a Skin Contact Time of at least 60 seconds.

8. The absorbent article of claim 7 wherein said portion has an area of at least 10 cm².

9. The absorbent article of claim 8 wherein said sensation member includes a waist portion wherein said waist portion is foreshortened at a Contraction Ratio at a 10 g load of less than about 0.2.

10. The absorbent article of claim 9 wherein said foreshortened waist portion is stretchable from a relaxed state to a fully elongated state and wherein the force required to elongate said foreshortened region of said sensation member from a relaxed state to 85% of its length in a fully elongated state is at least 0.4 N.

11. A refastenable absorbent article having a waist region and a crotch region, the article comprising:
an outer cover, wherein said outer cover has a moisture vapor transmission rate of at least 1000 g/m²/24 hr;
a sensation member having a body-facing surface, wherein at least a portion of the body facing surface of said sensation member is capable of contacting the skin of a wearer and wherein said portion has a Wet Peel Force of at least 30 grams force; and
an absorbent core disposed between the outer cover and the sensation member.

12. The absorbent article of claim 11 wherein said portion has a Wet Peel Force of at least 40 grams force.

13. The absorbent article of claim 12 wherein said portion has an area of at least 10 cm².

14. The absorbent article of claim 13 wherein said sensation member includes a waist portion wherein said waist portion is foreshortened at a Contraction Ratio at a 10 g load of less than about 0.2.

15. The absorbent article of claim 14 wherein said foreshortened waist portion is stretchable from a relaxed state to a fully elongated state and wherein the force required to elongate said foreshortened region of said sensation member from a relaxed state to 85% of its length in a fully elongated state is at least 0.4 N.

16. An array of absorbent articles comprising:
a first absorbent article comprising:
an outer cover;
a sensation member having a body-facing surface, wherein at least a portion of the body facing surface of said sensation member is capable of contacting the skin of a wearer and wherein said portion has a first property chosen for at least one of surface area, Contraction Ratio at a 10 g load, force required to elongate a foreshortened region of the sensation member from a relaxed state to 85% of its length in a fully elongated state, Stiffness, Skin Contact Time and Wet Peel Force; and
an absorbent core disposed between the outer cover and the sensation member; and
a second absorbent article comprising:
an outer cover;
a sensation member having a body-facing surface, wherein at least a portion of the body facing surface of said sensation member is capable of contacting the skin of a wearer and wherein said portion has a second property chosen for at least one of surface area, Contraction Ratio at a 10 g load, force required to elongate a foreshortened region of the sensation member from a relaxed state to 85% of its length in a fully elongated state, Stiffness, Skin Contact Time and Wet Peel Force; and
an absorbent core disposed between the outer cover and the sensation member wherein said first property of said first absorbent article has a different value than said second property of said second absorbent article.

17. The array of absorbent articles of claim 16 wherein said first and second absorbent articles are gender specific.

18. The array of absorbent articles of claim 16 wherein said first and second absorbent articles have different sizes.

19. An absorbent article having a waist region and a crotch region, the article comprising:
an outer cover, wherein said outer cover has a moisture vapor transmission rate of at least 1000 g/m²/24 hr;
first and second barrier leg cuffs, at least one of said first and second barrier leg cuffs having an upper edge;
a sensation member having a suspended central region having at least a side edge; and an absorbent core disposed between the outer cover and the sensation member,
wherein the projected height of the side edge is between about 90% and about 300% the projected height of the upper edge of at least one of said first and second barrier leg cuff.

20. The absorbent article of claim 19 wherein said suspended central region has a body facing surface wherein at least a portion of the body facing surface is capable of contacting the skin of a wearer.

21. The absorbent article of claim 20 wherein said portion has a Stiffness of less than about 100 N/m.

22. The absorbent article of claim 22 wherein said portion has a Skin Contact Time of at least 30 seconds.

23. The absorbent article of claim 22 wherein said portion has a Wet Peel Force of at least 30 grams force.

24. An absorbent article having a waist region and a crotch region, the article comprising:
an outer cover, wherein said outer cover has a moisture vapor transmission rate of at least 3500 g/m²/24 hr;
a sensation member having a body-facing surface, wherein at least a portion of the body facing surface of said sensation member is capable of contacting the skin of a wearer and wherein said portion has a Stiffness of less than about 100 N/m and wherein said portion of the body facing surface of the sensation member has a Skin Contact Time of at least 30 seconds; and
an absorbent core disposed between the outer cover and the sensation member.

25. The absorbent article of claim 24 wherein said portion has a Stiffness of less than about 75 N/m.

26. The absorbent article of claim 25 wherein said portion has an area of at least 10 cm².

27. The absorbent article of claim 25 wherein said sensation member includes a waist portion wherein said waist portion is foreshortened at a Contraction Ratio at a 10 g load of less than about 0.2.

28. The absorbent article of claim 27 wherein said foreshortened waist portion is stretchable from a relaxed state to a fully elongated state and wherein the force required to elongate said foreshortened region of said sensation member from a relaxed state to 85% of its length in a fully elongated state is at least 0.4 N.

29. The absorbent article of claim 24 wherein said portion has a Wet Peel Force of at least 30 grams force.
